(19) United States
(12) Patent Application Publication
(10) Pub. No.: US 2021/0335451 A1
(43) Pub. Date: Oct. 28, 2021

(54) TRANSCRIPTOMIC SIGNATURE FOR THE PROGNOSIS AND TREATMENT SELECTION FOR CERVICAL CANCER

(71) Applicant: Augusta University Research Institute, Inc., Augusta, GA (US)

(72) Inventor: Jin-Xiong SHE, Augusta, GA (US)

(73) Assignee: Augusta University Research Institute, Inc., Augusta, GA (US)

(21) Appl. No.: 17/240,386

(22) Filed: Apr. 26, 2021

Related U.S. Application Data
(60) Provisional application No. 63/015,045, filed on Apr. 24, 2020.

(51) Int. Cl. G16B 25/10; C12Q 1/6886; G16B 40/00; G16H 50/30; G16H 50/20; G16H 10/40; G16H 20/40; G06N 20/00

(52) U.S. Cl. CPC G16B 25/10; C12Q 1/6886; G16B 40/00; G16H 50/30; G16H 50/20; C12Q 2600/106; G16H 20/40; G06N 20/00; C12Q 2600/158; C12Q 2600/112; G16H 10/40

(57) ABSTRACT

Disclosed herein are methods of staging, treating and making prognostic prediction of, monitoring of therapeutic outcome for treatment of cervical carcinoma in a patient in need thereof by quantifying gene expression in a sample, wherein the genes include 40 high risk genes; calculating the subject's survival risk score by determining the gene expression levels and their inter-dependence using machine learning (ML) and artificial intelligence. The survival risk category of a patient is determined by the consensus or plurality voting of a large number of ML models that individually have excellent predictive potential, thus providing a very robust prognostic biomarker for cervical carcinoma.

### CROSS REFERENCE TO RELATED APPLICATIONS

This application claims benefit of and priority to U.S. Provisional Patent Application No. 63/015,045 filed on Apr. 24, 2020, and is incorporate by reference in its entirety.

### TECHNICAL FIELD OF THE INVENTION

This invention is generally related to cancer diagnostic methods and uses thereof.

### BACKGROUND OF THE INVENTION

Worldwide, cervical cancer is the most common and deadliest gynecologic malignancy, accounting for an estimated 570,000 new cases and 311,000 deaths each year (Bray, et al., CA Cancer J Clin., 68:394 (2018)). Despite efforts in screening and human papillomavirus (HPV) vaccine adoption, cervical cancer remains a persistent health challenge for women in the United States, with 13,170 new cases and 4,250 deaths estimated for 2019 (Siegel, et al., CA Cancer J Clin., 69:7 (2019)). Survival for women with cervical cancer has not significantly improved since the mid-1970s, in contrast to the majority of other common cancers in the United States (Jemal, et al., J Natl Cancer Inst., 109 (2017)). While early-stage cervical cancer can be successfully treated, with 5-year overall survival (OS) rates as high as 97%, metastatic cervical cancer is virtually incurable, with 5-year OS rates below 10% (Quinn, et al., Int J Gynae-col Obstet., 95 Suppl 1:S43-103 (2006)). For patients with recurrent cervical cancer, their prognosis remains poor. The mortality risk for metastatic or recurrent cervical cancer is high, with median OS remaining limited to less than 1.5 years, even with the 3.5 month gain in median OS shown in GOG 240 by adding bevacizumab to first-line systemic platinum-based combination chemotherapy (Tewari, et al., N Engl J Med., 370:734 (2014); Tewari, et al., Lancet 390: 1654 (2017)). Therefore, new approaches are needed to better identify and treat patients with cervical cancer at high risk of recurrence and death.

A major focus in improving systemic treatment of cervical cancer involves developing a better understanding of the genomic, transcriptomic, and proteomic underpinnings and heterogeneity of the disease. The central tenet in the pathogenesis of cervical cancer is the involvement of HPV, which can be found in up to 99.7% of cervical cancers (Walboomers, et al., J Pathol., 189:12 (1999)). Despite the near-universal contribution of HPV to cervical carcinogenesis, there is wide variance in the risk of cancer associated with the different types of carcinogenic HPV, as well as the association of types of carcinogenic HPV with the different histologic subtypes (squamous cell carcinoma and adenocarcinoma) of cervical cancer (Li, et al., Int J Cancer, 128:927 (2011)).

To further advance the molecular understanding of cervical cancer, The Cancer Genome Atlas (TCGA) project recently published their analysis of 228 primary cervical cancers (Cancer Genome Atlas Research Network, Nature, 543:378 (2017)). While the results from that project noted a number of novel molecular features, the integrated clustering, which identified 3 main subgroups (keratin-low squamous, keratin-high squamous, adenocarcinoma), was not based on patient outcomes such as survival. A proteomic grouping was associated with differences in survival, but that grouping was (a) not primarily based on patient outcomes and (b) used as a small component of the integrative clustering that resulted in the featured novel subgroups (of note, the prognostic value of the proteomic grouping was recently validated by a separate group and dataset (Rader, et al., Gynecol Oncol., 155:324 (2019)). Further, no data was reported by TCGA to show that differences in the main novel cervical cancer subgroups were associated with differences in clinically relevant outcomes. Several other studies have investigated the genomic contributions to differences in clinical outcomes in cervical cancer, but outcomes were typically not a starting point in those studies, and their sample sizes were much smaller than TCGA (Barron, et al., PLoS One, 10:e0137397 (2015); Espinosa, et al., PLoS One, 8:e55975 (2013); Medina-Martinez, et al., PLoS One, 9:e97842 (2014); Wright, et al., Cancer, 119:3776 (2013)). Other groups have evaluated the potential of micro-RNA signatures for use as prognostic biomarkers, but results have been mixed and the most promising of those signatures did not validate (How, et al., PLoS One, 10:e0123946 (2015); Liu et al., Oncotarget, 7:56690 (2016); Zeng et al., J Cell Biochem, 119:1558-1566 (2018)). Further, it is unclear whether the findings in above studies were confounded by fundamental differences between the 2 major histologic subtypes of cervical cancer (squamous cell vs. adenocarcinoma), which arise from separate sites of the cervix and have different molecular profiles (Wright, et al., Cancer, 119:3776 (2013)).

Locally advanced cervical cancer can be treated with surgery, radiation, chemoradiation (CRT), or a combination of these modalities (Rose et al. 1999, Cohen et al. 2019). These options work well in patients who have localized disease as the 5-year survival is 85 to 90% (Cohen et al. 2019, Kim, Choi, and Byun 2000). One challenge for clinicians is determining which patients will need adjuvant treatment following surgery, as the use of dual modality therapy has been associated with considerable morbidity (Peters III et al. 2000, Sedlis et al. 1999). Therefore, the decision to recommend surgery or CRT is multifactorial, taking into account comorbidities, available pathologic and imaging data, and the side effect profiles of the different treatment modalities (Landoni et al. 2017, Vistad, Fossa, and Dahl 2006). A prognostic score capable of predicting survival and treatment response would greatly help in counseling patients on potential options at the time of diagnosis or after surgery.

To date, there have been few genetic scores developed for cervical cancer (Wong et al. 2003, Wang et al. 2019, Huang et al. 2012). These prior studies have been limited by sample size, lack of validation, or no association with treatment response.

Therefore, there is a need to identify sets of genes that can identify subgroups with large and clinically meaningful survival differences and to develop a genetic risk score capable of predicting prognosis and stratify patients into those who will or will not respond to primary therapy.

It is an object of the invention to provide methods and reagents for diagnosis or assisting in the prognosis of cancer.

### SUMMARY OF THE INVENTION

Survival for patients with newly diagnosed cervical cancer has not significantly improved over the past several decades. Disclosed herein is a clinically relevant set of prognostic genes for squamous cell carcinoma of the cervix (SCCC), the most common cervical cancer subtype. Using RNA-sequencing data and survival data from 203 patients in The Cancer Genome Atlas (TCGA), a series of analyses using different decile and quartile cutoffs for gene expression was performed to identify genes that could indicate large and consistent survival differences across different cutoffs of gene expression. Those analyses identified 40 prognostic genes that have the greatest utility to stage cervical cancer and include the following: EGLN1, CD46, PLOD1, QSOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145, and combinations thereof. In one embodiment, a patient's survivability is estimated by using gene expression levels of each of the individual 40 genes and more importantly by using a machine learning (ML) algorithm such as Ridge regression to calculate a Ridge regression score, wherein a smaller Ridge regression score indicates lower survivability than a larger score. Other machine learning methods can also be used to calculate a transcriptomic risk score similar to Ridge Regression Score. In some embodiments, a Ridge regression score is calculated by using any combination of two to thirty-nine genes or all forty genes disclosed above. In some embodiments the RNA gene expression of 2, 5, 10, 15, 20, 25, 30, 35 or all 40 genes is used to calculate the subject's Ridge regression score. In one embodiment, large numbers of Ridge regression models can be created by randomly sampling gene expression of the 40 genes of subsets of patients from all patients in the total data. In still another embodiment, the final staging of a patient is determined by the consensus of two or more Ridge regression models created using the expression levels of any combination of the forty genes disclosed above. This transcriptomic biomarker can better predict survival than clinical prognostic factors, including the stage of the cancer in the subject.

One embodiment provides method of assessing a patient's survivability by determining RNA levels of one or more of the genes selected from the group consisting of EGLN1, CD46, PLOD1, Q SOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145, and subcombinations thereof from a sample from the patient and comparing the patient's RNA levels to RNA levels of reference samples with a known survivability assignment. In some embodiments the RNA gene expression of 1, 2, 5, 10, 15, 20, 25, 30, 35 or all 40 genes is used to generate a survivability assignment or transcriptomic risk scores (TRS). Gene expression levels can be determined using RT-PCR, microarrays, RNAseq, or other standard molecular biology technique. The method also includes generating multigenic models using modeling techniques including, but not limited to, machine learning such as Ridge regression and deep learning to compute transcriptomic risk scores for patients. The transcriptomic risk scores are then used to stratify patients into low, intermediate, and high TRS groups using a using a plurality voting of the models. In some embodiments the models are predictive models for predicting the survivability of the patient. The disclosed methods can be used to estimate survival time of the patient, estimate treatment outcome, inform decisions on therapeutic options, and assist in the selection of new therapies versus traditional therapies. For a patient with a high risk score, more aggressive treatment would be selected for the patient.

One embodiment provides a method for staging a patient's cervical cancer by using gene expression levels in one or more of the following genes or any combination thereof: EGLN1, CD46, PLOD1, QSOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145, and using Ridge regression, a machine learning (ML) algorithm, to calculate a Ridge regression score for the patient, wherein the Ridge regression score is compared to a Ridge regression score of patients with a known stage of cervical cancer to stage the patient's cervical cancer. In some embodiments the RNA gene expression of 2, 5, 10, 15, 20, 25, or all 40 genes is used to calculate the subject's Ridge regression score. In one embodiment, thousands or more Ridge regression models can are created by randomly sampling subsets of patients from all patients in the total data. In still another embodiment, the final staging of a patient is determined by the consensus of 2 or any number of models greater than 2 Ridge regression models created using subsets of patients and subsets of the 40 genes disclosed above.

Another embodiment provides a method of prognosing and treating cervical carcinoma in a subject in need thereof by quantifying RNA gene expression in a sample, wherein the genes include one or more of the 40 genes or any combination thereof selected from the group consisting of EGLN1, CD46, PLOD1, QSOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145; calculating the subject's Ridge regression score (RRS) by examining the expression levels of the genes and the inter-dependence between the two or more of the 40 genes, wherein a higher TRS indicates that the patient may not respond to therapy. The method further includes the step of administering radiation and/or chemotherapy to the patient diagnosed with cervical carcinoma. In some embodiments the RNA gene expression of 1, 2, 5, 10, 15, 20, 25, or all 40 genes is used to calculate the subject's TRS. The groups of genes used can be in any combination of the 40 genes disclosed above in groups of 2, 3, 4, 5, 6, 7, 8, 9, 10, 11, 12, 13, 14, 15, 16, 17, 18, 19, 20, 21, 22, 23, 23, 25, 30, 35, and 40.

Still another embodiment provides a method of prognosing and treating cervical carcinoma in a subject in need thereof by generating different machine learning models (ML models) using gene expression of two or more genes selected from the group consisting of EGLN1, CD46, PLOD1, Q SOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145, and combinations thereof from subsets of patients in a dataset and the plurality voting of the top models to calculate an TRS, wherein a higher TRS indicates that the patient has worse survivability than a lower TRS. The method further includes the step of administering radiation and/or chemotherapy to the patient diagnosed with cervical carcinoma.

One embodiment provides a method of diagnosing and treating cervical carcinoma in a subject in need thereof by generating different machine learning models (ML models) using gene expression of one or more genes selected from the group consisting of EGLN1, CD46, PLOD1, QSOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145, and combinations thereof from subsets of patients in a dataset and the plurality voting of the top models to calculate an TRS for each model, wherein an TRS is associated with survivability of cervical carcinoma. The method further includes the step of administering radiation and/or chemotherapy to the patient having diagnosed with cervical carcinoma. In some embodiments the RNA gene expression of 2, 5, 10, 15, 20, 25, 30, or all 40 genes is used to calculate the subject's TRS. The groups of genes used can be in any combination of the 40 genes disclosed above for example any combination of genes in groups of 2, 3, 4, 5, 6, 7, 8, 9, 10, 11, 12, 13, 14, 15, 16, 17, 18, 19, 20, 21, 22, 23, 23, 25, 30, 35 and 40.

One embodiment provides a method of diagnosing and treating cervical carcinoma in a subject in need thereof by developing a transcriptomic risk score (TRS) from expression data of one or more of the genes selected from the group consisting of EGLN1, CD46, PLOD1, QSOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145, and combinations thereof from which predicts prognosis and stratifies patients into those who will respond well or poorly to primary therapy by calculating an TRS. In some embodiments, the TRS identifies patients who may not need aggressive therapies such as chemotherapy or radiation therapy; and the TRS also identifies patients who do not respond well to primary therapy and need therapies with better efficacy. In some embodiments the RNA gene expression of 2, 5, 10, 15, 20, 25, 30, 35 or all 40 genes is used to calculate the subject's TRS. The groups of genes used can be in any combination of the forty genes disclosed above.

Another embodiment provides a method for identifying biological pathways that can be targeted to improve the poor prognosis of those patients with disease predicted to be unresponsive to chemotherapy and radiation therapy. For example, one or more of the 40 genes recited above can be targeted to modulate their expression to improve a poor prognosis of a patient.

### BRIEF DESCRIPTION OF THE DRAWINGS

FIGS. 1A-1F are representative Kaplan-Meier survival curves (6 shown) for the top 40 prognostic genes. X-axis: time (years); Y-axis: survival probability.

FIGS. 2A-2L are survival curves for four representative models (each model in a row) based on the expression levels of the 25 or 20 top genes. Survival was assessed in train datasets (50% of the patients) and the test dataset (the remaining 50% of the patients) as well as the entire dataset (third column).

FIG. 3 is a heatmap showing the votes for each of the 203 patients (row) by each of the 80 top models (column). Patients were ordered by the increasing percentage of votes for the RRS high group.

FIGS. 4A-4D are Kaplan-Meier survival curves for patient subsets assigned based on plurality voting of the top 80 Ridge regression models. Patients in the RRS_L group received at least 75% of the votes for the low group and patients in the RRS_H group received at least 75% of the votes for the high group, while the patients in the RRS_M group received 25-75% of votes for the low group. 4A & 4B: All patients were included. Patients were classified in three groups in 4A while low- and moderate-risk groups were combined in 4B. 4C & 4D: Stage 4 patients were excluded in analyses. Patients were classified in three groups in 4C while low- and moderate-risk groups were combined in 4D.

FIGS. 5A-5C are survival curves for the major clinical oncologic variables for SCCC. X-axes=time (years).

### DETAILED DESCRIPTION OF THE INVENTION

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### I. Transcriptomic Signature for the Prognosis and Treatment Selection for Cervical Cancer

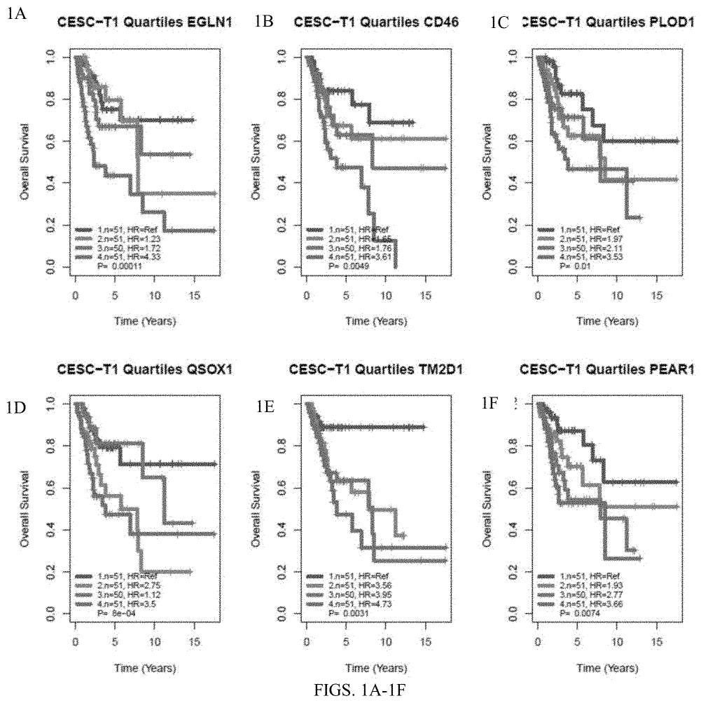

Disclosed herein is a clinically relevant set of prognostic genes for squamous cell carcinoma of the cervix (SCCC), the most common cervical cancer subtype. Forty (40) genes were identified that individually predict SCCC patient survival (FIG. 1). The majority of identified genes have been associated with key cancer hallmarks such as cellular proliferation, migration/invasion, and/or metastasis. The survival prognosis appears to be influenced not only by the expression level of each high-risk gene but also the number of the genes with the highest expression levels. Survival gradually worsened as expression level of the 40 genes increased (FIG. 1). Poorest survival was observed in patients with highest expression for 5 or more genes; best survival was observed in patients with fewer genes with highest expression. These results suggest that the risk for dying of SCCC is determined by the patient's transcriptomic risk burden. Machine learning identified gene signatures that are sufficiently accurate to predict survival. Transcriptomic risk scores for mortality can be computed with the pronostic genes utilized to stratify patients into low, intermediate, and high TRS groups. Based on the analyses of TCGA SCCC patient population, while stage IV was a very good predictor for poor survival, TRS was not entirely confounded by stage or any other clinical variables. Indeed, multivariate analyses using TRS and the prognostically significant clinical parameters for SCCC demonstrated that TRS was by far a better survival predictor than stage. And even in a patient population that did not have a significant survival difference among stages I-III, TRS could identify patients at high, intermediate, and low risk of mortality.

In current clinical practice, there is no prognostic biomarker for cervical cancer. Factors that inform adjuvant treatment for early cervical cancer include: a risk stratification based on stromal invasion, lymphovascular space invasion, and tumor diameter (Sedlis, et al., Gynecol Oncol., 73:177 (1999)) (intermediate-risk disease: give pelvic radiotherapy); criteria for high-risk of recurrence and death (positive margins, positive lymph nodes, parametrial involvement) that merit chemoradiation (Peters, et al., J Clin Oncol., 18:1606-1613 (2000)). For locally advanced cervical cancer, chemoradiation is standard of care; the benefit of additional chemotherapy given after chemoradiation is currently under investigation (ClinicalTrials.gov Identifier: NCT01414608). Stage and lymph node status can influence treatment planning for cervical cancer, but those factors may miss some patients at high risk for mortality.

Data from presented herein raises the concern that early stage may underestimate mortality in some patients, as approximately 40.9% of early stage (stage I and II) patients in the studied TCGA SCCC population were high TRS and poor survivors. Given the finding that TRS appears to outperform stage and lymph node status as a prognostic variable, it warrants further investigation as a biomarker for SCCC. Such would be especially important to a poor-prognosis subgroup of earlier-stage SCCC patients with high TRS, who might be under-treated relative to their prognosis based on clinical factors alone.

Another important observation was that 47.8% of late stage (stage III and IV) SCCC had low TRS associated with good survival, which would suggest that a subset of late stage SCCC patients may have an overestimation of mortality risk with clinical factors alone. Further investigation in more patients would be needed to confirm the presence and degree of prognosis-modifying impact of low TRS in patients with stage III SCCC. However, the finding of 2 within-stage TRS subgroups prognostically different than expected based on stage alone strongly suggests that TRS is not completely confounded by stage.

This disclosure also provides a new perspective on gene expression in SCCC with respect to survival. The approach taken is quite different from prior studies in several respects: clinical outcomes were not a starting point in those studies, sample sizes were much smaller than TCGA, analysis was limited to specific gene types (e.g., micro-RNAs), and/or the inclusion of both major histologic subtypes may have confounded the genomic analyses (Cancer Genome Atlas Research Network, Nature, 543:378 (2017); Barron, et al., PLoS One, 10:e0137397 (2015); Espinosa, et al., PLoS One, 8:e55975 (2013); Medina-Martinez, et al., PLoS One, 9:e97842 (2014); Wright, et al., Cancer, 119:3776 (2013); How, et al., PLoS One, 10:e0123946 (2015); Liu et al., Oncotarget, 7:56690 (2016); Zeng et al., J Cell Biochem, 119:1558-1566 (2018)). In contrast, this work leveraged the relatively high number of SCCC patients with both gene expression and survival data and avoided the pitfalls of grouping multiple histologic subtypes into a single-omic analysis. Further, an analysis was conducted through the lens of clinical relevance (i.e., who survived and who died?). While the finding of a transcriptomic risk gene signature for SCCC has not yet been validated with a separate data set, a strength of this disclosure is its focus on genes showing large and consistent survival differences at multiple cutoffs. Such genes are more likely to be validated in other datasets and be clinically relevant.

One innovation of this work is the discovery and selection of a large number of prognostic models based on machine learning. This is achieved by sampling thousands of subsets of patients for training the models and using the remaining samples for testing the models. Only those models that provide excellent prognostic potential in both training and testing are kept and used for prognostic prediction. Furthermore, selected models are validated by a bootstrapping procedure.

Another innovation is to use plurality voting or consensus of many excellent machine learning models to determine the final assignment of each patient to different survival groups. This innovative approach results in a biomarker that is much more likely to applicable to future samples because the classification is not based on any individual genes or any individual models.

40 genes were found that are highly associated with survival in SCCC (Table 5). Among TCGA SCCC patients analyzed, survival prognosis worsened with (a) increasing expression level for each individual high-risk gene or (b) a greater number of those genes with high expression level in a patient's tumor. These findings suggest the importance of the transcriptomic risk load on survival. The plurality voting of ML models appear to have better prognostic ability than any reported prognostic marker for SCCC, including stage and lymph node status. Although the clinical application of these discoveries will require validation in other datasets, this disclosure provides a roadmap towards a clinically meaningful prognostic biomarker for SCCC.

A. 25 and 20 Gene Signatures

To generate models for survival prediction, the entire dataset was randomly divided into training and testing subset, each containing 50% of the patients. This process was repeated 3000 times to generate 3000 pairs of training and testing subsets of patients. For each subset of patients, Ridge regression analysis was carried out to calculate a ridge regression score (RRS) using all 40 genes and the relative contribution of each gene to RRS in the first round of analyses. Proportional hazard analysis for survival was carried out to calculate hazard ratio (HR) and p value using RRS. All 3000 models were evaluated using their HRs in the training and testing pair. The HRs were greater than 5 in both the training and testing subsets for 86 models, which were then used to rank the 40 genes based on the mean contribution of the gene to the RRS of all 86 top models. The top 25 genes were selected and shown in Table 1.

TABLE 1
Ridge regression scores for the top
40, 25 or 20 genes in the top models

| | Gene | | | |
|---|---|---|---|---|
| Order | Name | RRS_40 | RRS_25 | RRS_20 |
| 1 | TM2D1 | 0.16 | 0.17 | 0.21 |
| 2 | EGLN1 | 0.15 | 0.16 | 0.20 |
| 3 | PLK1S1 | 0.11 | 0.11 | 0.14 |
| 4 | CD46 | 0.10 | 0.11 | 0.13 |
| 5 | SDF4 | 0.10 | 0.12 | 0.14 |
| 6 | PEAR1 | 0.09 | 0.10 | 0.12 |
| 7 | AIMP2 | 0.08 | 0.09 | 0.13 |
| 8 | MMS19 | 0.07 | 0.08 | 0.12 |
| 9 | PLOD1 | 0.07 | 0.09 | 0.10 |
| 10 | BRSK1 | 0.07 | 0.08 | 0.11 |
| 11 | NRP1 | 0.07 | 0.09 | 0.12 |
| 12 | GALNT3 | 0.06 | 0.07 | 0.09 |
| 13 | LIF | 0.06 | 0.06 | 0.08 |
| 14 | PFKP | 0.06 | 0.08 | 0.10 |
| 15 | QSOX1 | 0.06 | 0.06 | 0.07 |
| 16 | ZNF701 | 0.06 | 0.05 | 0.06 |
| 17 | GPR27 | 0.06 | 0.07 | 0.09 |
| 18 | ANGPTL4 | 0.05 | 0.07 | 0.08 |
| 19 | GALNT2 | 0.05 | 0.05 | 0.05 |
| 20 | FNDC3A | 0.05 | 0.04 | 0.06 |
| 21 | TMED4 | 0.05 | 0.05 | |
| 22 | PRR12 | 0.05 | 0.05 | |
| 23 | APCDD1L | 0.04 | 0.05 | |
| 24 | MTDH | 0.04 | 0.05 | |
| 25 | GRB10 | 0.04 | 0.04 | |

RRS_40: mean ridge regression score in the top 40-gene models
RRS_25: mean ridge regression score in the top 25-gene models
RRS_20: mean ridge regression score in the top 20-gene models

1. Ridge Models with the Top 25 and Top 20 Genes

Three thousand (3000) random training/testing pairs were generated and Ridge regression analyses conducted with the 25 top genes. A total of 190 models yielded HRs greater than 5 in both the training and testing subsets, more than doubling the number of models with comparable performance from the 40 gene models, suggesting that the top 25 genes perform much better than the top 40 genes. Data for 40 selected models are shown in Table 2. Furthermore, 3000 Ridge regression models were developed and assessed with the top 20 genes and 245 models had HRs >5 in both training and testing, tripling the number of excellent models from the 40-gene analysis but only modestly higher than the 25-gene models. As quality control for the analytical pipeline, 3000 models were also developed and assessed using the bottom 20 genes that were not used in the top 20 gene analyses. Interestingly but not surprisingly, none of the 3000 bottom 20-gene models yielded HRs >5 in both training and testing; and only 28 models had HR >3 in both training and testing. These results suggest that the gene selection pipeline presented herein performs very well.

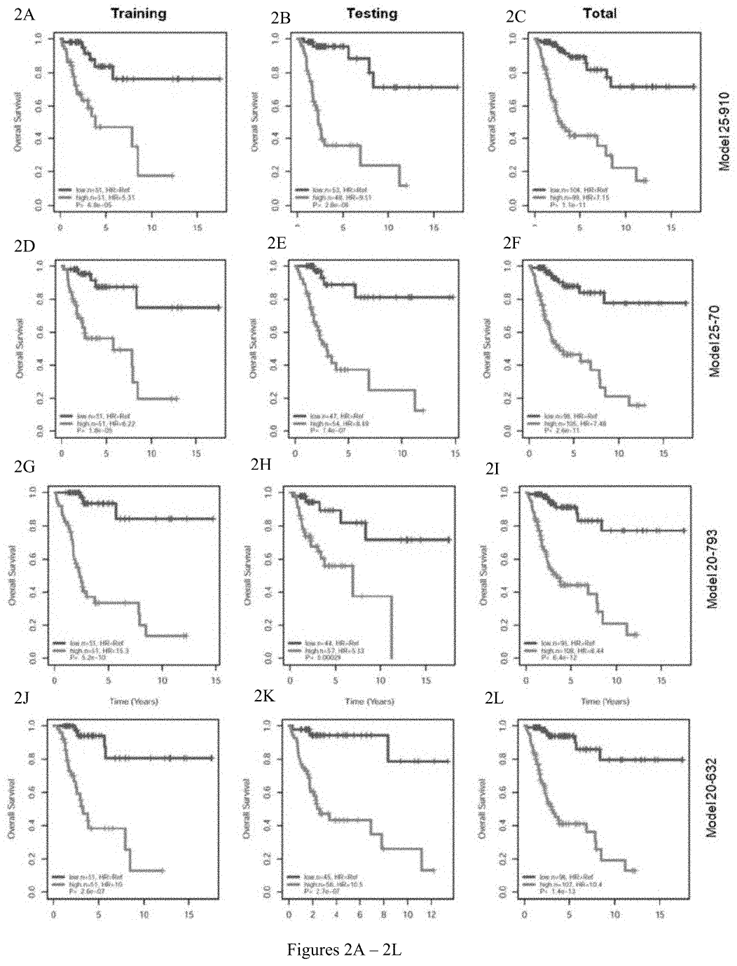

FIGS. 2A-2L shows the Kaplan-Meier (KM) survival curves for four representative models in the training and testing subsets as well as in the entire dataset. Patients with low RRS have approximate 80% survival at 10 years and beyond compared to approximate 20% survival at 10 years for patients with high RRS.

2. Consensus Model

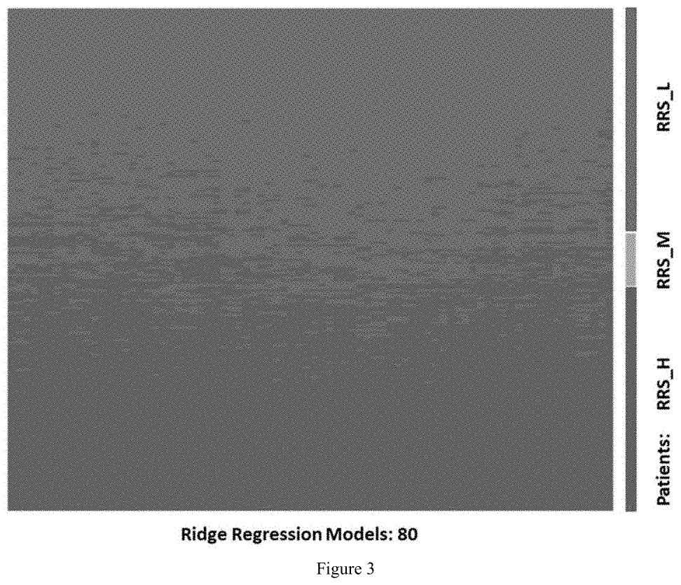

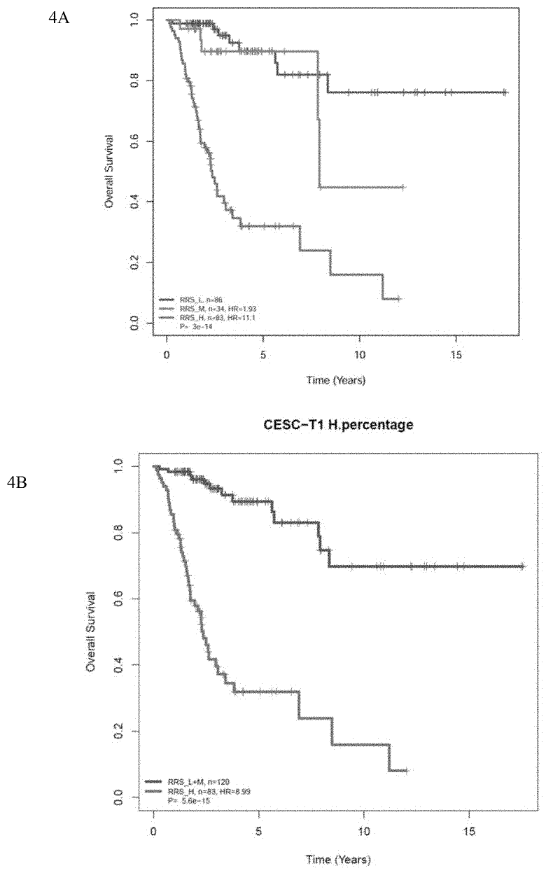

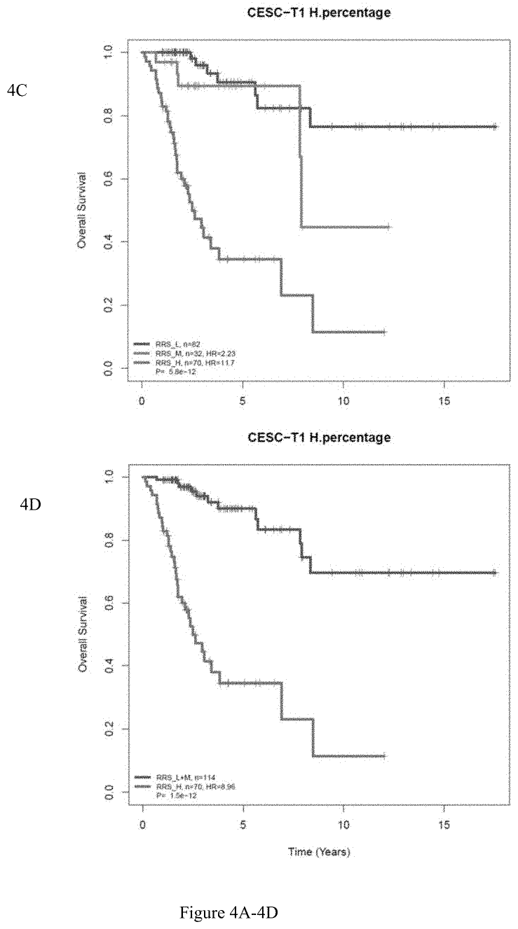

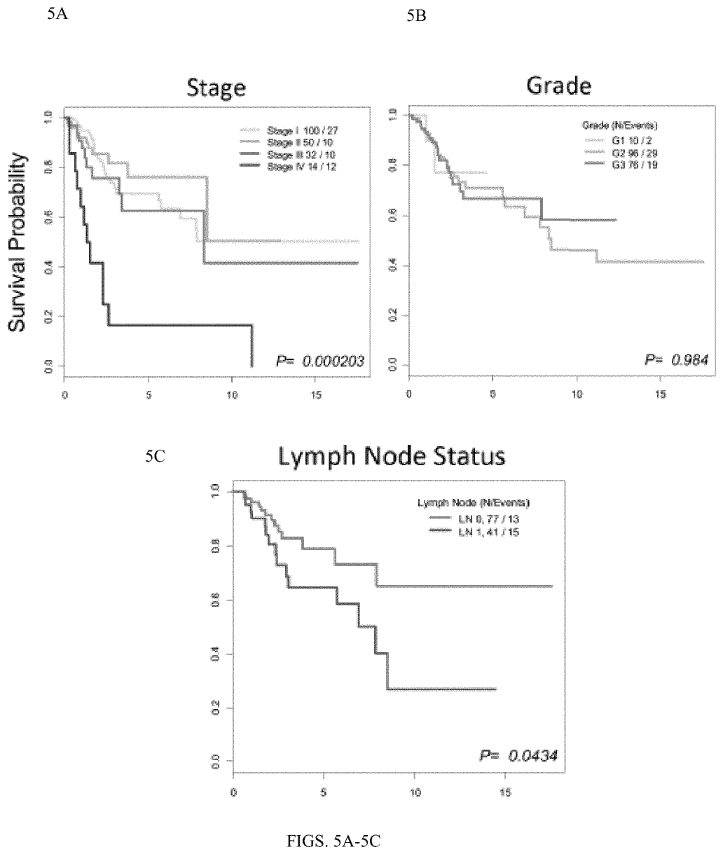

Whether and how consistently different Ridge regression models classify each patient to the RRS groups was examined next. For this purpose, the top 40 models from the 25-gene analysis and top 40 models from the 20-gene analysis were selected based on the ranking of mean HRs from the bootstrapping analyses (Table 2) to examine how each of the 80 models vote on the classification of each patient. As shown in FIG. 3, at least 75% of the models voted 86 patients to the RRS_low group and at least 75% of models voted 83 other patients to RRS-high group, while 50-75% of the models voted the remaining 34 patients to RRS_low (this was called the Middle or ambiguous group). These results suggest that the vast majority of the patients can be confidently assigned to the RRS_low or RRS_high groups. Survival differs dramatically between the RRS_low and RRS_high groups (HR=11.1, p<3E-14) (FIG. 4A). While the middle group with a small proportion of patients has very similar survival as the RRS_low group in the current dataset (FIG. 4A), the classification and prognosis for these patients are less confident. Consistent with the data from single Ridge models, the consensus of 80 models indicates that RRS_low patients (42.4% of the total patients) have approximately 80% survival at 10 years and beyond compared to less than 20% survival at 10 years for patients with high RRS.

One embodiment provides a method of diagnosing and treating cervical carcinoma in a subject in need thereof by quantifying RNA gene expression in a sample, wherein the genes include the 40 genes listed in Table 1; calculating the subject's survival risk score by determining the expression level of genes and their relationships;

One embodiment provides a method of diagnosing and treating cervical carcinoma in a subject in need thereof by quantifying RNA gene expression in a sample, wherein the genes include one or more of the 40 genes listed in Table 1; calculating the subject's Ridge regression score (RRS) by examining the expression levels of genes and the relationships between two to forty different genes;

One embodiment provides a method of diagnosing and treating cervical carcinoma in a subject in need thereof by generating different ML models using subsets of patients in a dataset and the plurality voting of the top models;

One embodiment provides a method of diagnosing and treating cervical carcinoma in a subject in need thereof by developing a transcriptomic risk score (TRS) or Ridge regression score (RRS) capable of predicting prognosis and stratify patients into those who will respond well or poorly to primary therapy. Furthermore, the TRS/RRS would also help identify patients who may not need aggressive therapies such as chemotherapy or radiation therapy; and TRS/RRS would help identify patients who do not respond well to primary therapy and need therapies with better efficacy.

Another embodiment provides insight towards potential pathways which could be targeted to improve the poor prognosis of those patients with disease predicted to be unresponsive to chemotherapy and radiation therapy.

TABLE 2
Top Ridge regression models and their performances for survival prediction.

| | | | | | | Bootstrapping | | | | | |
|---|---|---|---|---|---|---|---|---|---|---|---|
| Gene | Model | | Training testing 50% split | | | | | | p > | p .05 | p < |
| Set | # | HR_train | p_train | HR_test | p_test | HR_mean | HR_low | HR_up | 0.05 | to E−6 | E−6 |
| 25 | 910 | 5.31 | 6.84E−05 | 9.51 | 2.75E−08 | 8.51 | 5.1 | 15.5 | 0 | 32 | 968 |
| 25 | 70 | 6.22 | 1.84E−05 | 8.49 | 7.38E−07 | 8.23 | 5.15 | 14.81 | 0 | 43 | 957 |
| 25 | 473 | 13.5 | 6.57E−09 | 5.01 | 9.42E−05 | 8.18 | 5.26 | 13.86 | 0 | 33 | 967 |
| 25 | 835 | 8.13 | 4.03E−07 | 7.27 | 3.37E−06 | 8 | 5.12 | 13.16 | 0 | 46 | 954 |
| 25 | 789 | 12.3 | 2.30E−09 | 5.11 | 6.05E−05 | 7.96 | 5.07 | 13.63 | 0 | 44 | 956 |
| 25 | 931 | 8.43 | 6.88E−07 | 6.8 | 1.23E−05 | 7.79 | 5.11 | 12.57 | 0 | 41 | 959 |
| 25 | 1000 | 9.48 | 4.02E−06 | 6.32 | 1.17E−06 | 7.78 | 4.97 | 12.37 | 0 | 53 | 947 |
| 25 | 630 | 8.43 | 2.81E−07 | 6.33 | 5.15E−06 | 7.62 | 5.02 | 12.23 | 0 | 65 | 935 |
| 25 | 614 | 5.27 | 5.22E−06 | 10.6 | 3.34E−07 | 7.53 | 4.92 | 11.76 | 0 | 65 | 935 |
| 25 | 193 | 5.53 | 3.66E−05 | 9.22 | 3.83E−07 | 7.44 | 4.82 | 12.41 | 0 | 80 | 920 |
| 25 | 544 | 5.91 | 1.09E−05 | 8.92 | 3.85E−06 | 7.31 | 4.65 | 11.69 | 0 | 78 | 922 |
| 25 | 351 | 5.68 | 2.11E−05 | 10.8 | 3.33E−07 | 7.3 | 4.84 | 12.51 | 0 | 96 | 904 |
| 25 | 897 | 7.39 | 1.35E−06 | 8.17 | 6.34E−07 | 7.29 | 4.56 | 12.6 | 0 | 105 | 895 |
| 25 | 447 | 9.65 | 4.72E−07 | 5.76 | 1.08E−05 | 7.26 | 4.49 | 12.26 | 0 | 115 | 885 |
| 25 | 964 | 11 | 8.51E−08 | 5.59 | 1.61E−05 | 7.19 | 4.6 | 12.19 | 0 | 94 | 906 |
| 25 | 176 | 6.64 | 1.29E−06 | 8.67 | 3.06E−07 | 6.97 | 4.67 | 10.82 | 0 | 123 | 877 |
| 25 | 529 | 9.73 | 1.73E−06 | 5.48 | 1.06E−05 | 6.92 | 4.53 | 11.09 | 0 | 140 | 860 |
| 25 | 383 | 10.9 | 6.21E−08 | 5.51 | 4.30E−05 | 6.9 | 4.26 | 11.74 | 0 | 144 | 856 |
| 25 | 264 | 6.25 | 4.14E−06 | 9.55 | 1.56E−06 | 6.88 | 4.45 | 10.73 | 0 | 99 | 901 |
| 25 | 434 | 6.19 | 4.54E−07 | 10.2 | 8.79E−07 | 6.83 | 4.39 | 11.26 | 0 | 148 | 852 |
| 25 | 605 | 6.47 | 3.07E−06 | 10.1 | 7.68E−07 | 6.81 | 4.34 | 11.3 | 0 | 152 | 848 |
| 25 | 950 | 10.6 | 3.70E−08 | 6.79 | 1.69E−06 | 6.8 | 4.23 | 11.16 | 0 | 150 | 850 |
| 25 | 21 | 10.3 | 7.03E−07 | 5.36 | 8.18E−06 | 6.77 | 4.25 | 11.1 | 0 | 163 | 837 |
| 25 | 115 | 9.4 | 1.24E−07 | 5.47 | 6.12E−05 | 6.69 | 4.34 | 10.43 | 0 | 150 | 850 |
| 25 | 246 | 8.98 | 1.14E−07 | 6.45 | 2.86E−05 | 6.69 | 4.26 | 10.52 | 0 | 179 | 821 |
| 25 | 172 | 8.6 | 7.18E−07 | 6.74 | 8.08E−07 | 6.66 | 4.32 | 10.81 | 0 | 174 | 826 |
| 25 | 202 | 9.72 | 3.04E−06 | 6.03 | 9.69E−07 | 6.61 | 4.34 | 10.96 | 0 | 168 | 832 |
| 25 | 607 | 9.33 | 2.76E−07 | 6.62 | 1.46E−06 | 6.5 | 4.01 | 10.68 | 0 | 247 | 753 |
| 25 | 27 | 9.36 | 5.39E−06 | 5.76 | 4.19E−06 | 6.46 | 4.33 | 10.36 | 0 | 204 | 796 |
| 25 | 763 | 9.36 | 2.56E−07 | 6.14 | 1.41E−05 | 6.3 | 3.95 | 10.43 | 0 | 297 | 703 |
| 25 | 647 | 12.2 | 1.66E−07 | 8.24 | 1.20E−05 | 6.26 | 4.12 | 9.71 | 0 | 214 | 786 |
| 25 | 710 | 5.64 | 2.49E−05 | 11.2 | 3.32E−07 | 6.22 | 4.19 | 9.71 | 0 | 217 | 783 |
| 25 | 999 | 9.22 | 1.07E−06 | 6.08 | 1.63E−05 | 6.11 | 4.06 | 9.65 | 0 | 265 | 735 |
| 25 | 255 | 9.63 | 3.16E−07 | 5.16 | 1.64E−05 | 6.11 | 3.83 | 10.14 | 0 | 331 | 669 |
| 25 | 82 | 15.4 | 5.95E−10 | 5.74 | 1.47E−05 | 6.08 | 3.85 | 10.49 | 0 | 331 | 669 |
| 25 | 761 | 9.9 | 1.56E−08 | 5.22 | 3.74E−05 | 5.99 | 3.83 | 9.74 | 0 | 319 | 681 |
| 25 | 764 | 6.8 | 6.73E−07 | 8.11 | 7.82E−07 | 5.95 | 3.93 | 9.52 | 0 | 334 | 666 |
| 25 | 656 | 10.6 | 1.59E−06 | 5.01 | 4.69E−05 | 5.77 | 3.86 | 8.84 | 0 | 375 | 625 |
| 25 | 330 | 12.1 | 1.29E−07 | 5.97 | 3.02E−07 | 5.77 | 3.93 | 9.09 | 0 | 379 | 621 |
| 25 | 799 | 5.42 | 1.71E−05 | 9.41 | 7.70E−08 | 5.77 | 3.61 | 9.35 | 0 | 431 | 569 |
| 20 | 632 | 10.0 | 2.64E−07 | 10.5 | 2.71E−07 | 9.6 | 5.9 | 16.8 | 0 | 6 | 994 |
| 20 | 70 | 8.8 | 1.24E−06 | 7.2 | 5.26E−07 | 8.6 | 4.6 | 15.8 | 0 | 52 | 948 |
| 20 | 793 | 15.3 | 5.25E−10 | 5.3 | 0.000288 | 8.3 | 5.3 | 14.0 | 0 | 25 | 975 |
| 20 | 351 | 5.8 | 2.03E−05 | 10.4 | 7.87E−07 | 8.1 | 5.1 | 14.3 | 0 | 51 | 949 |
| 20 | 277 | 9.8 | 2.04E−07 | 6.2 | 1.11E−05 | 8.0 | 5.2 | 13.2 | 0 | 37 | 963 |
| 20 | 208 | 9.5 | 1.50E−07 | 6.9 | 1.31E−06 | 7.9 | 5.1 | 13.3 | 0 | 47 | 953 |
| 20 | 630 | 8.4 | 2.81E−07 | 6.9 | 1.18E−06 | 7.6 | 4.9 | 12.7 | 0 | 55 | 945 |
| 20 | 838 | 13.2 | 5.01E−08 | 5.5 | 3.83E−06 | 7.5 | 5.1 | 12.3 | 0 | 55 | 945 |
| 20 | 245 | 7.3 | 8.12E−07 | 9.1 | 6.07E−05 | 7.4 | 4.9 | 11.7 | 0 | 52 | 948 |
| 20 | 434 | 7.2 | 5.85E−08 | 10.3 | 4.07E−06 | 7.3 | 4.8 | 12.0 | 0 | 68 | 932 |
| 20 | 57 | 9.6 | 8.87E−08 | 6.2 | 1.15E−05 | 7.3 | 4.6 | 11.6 | 0 | 112 | 888 |
| 20 | 532 | 9.0 | 1.46E−06 | 6.9 | 6.67E−07 | 7.3 | 4.7 | 11.7 | 0 | 57 | 943 |
| 20 | 173 | 8.6 | 9.92E−07 | 6.6 | 1.01E−05 | 7.2 | 4.5 | 11.9 | 0 | 93 | 907 |
| 20 | 728 | 11.1 | 7.72E−09 | 5.6 | 5.06E−05 | 7.2 | 4.5 | 11.9 | 0 | 113 | 887 |
| 20 | 952 | 8.8 | 7.84E−08 | 6.2 | 6.17E−05 | 7.2 | 4.3 | 11.8 | 0 | 118 | 882 |
| 20 | 341 | 10.0 | 1.24E−06 | 5.4 | 1.10E−05 | 7.0 | 4.5 | 11.5 | 0 | 159 | 841 |
| 20 | 875 | 11.4 | 2.35E−07 | 5.6 | 5.11E−06 | 7.0 | 4.6 | 11.4 | 0 | 136 | 864 |
| 20 | 851 | 8.8 | 7.62E−07 | 17.4 | 7.12E−06 | 6.8 | 4.4 | 11.0 | 0 | 153 | 847 |
| 20 | 710 | 5.1 | 6.18E−05 | 11.3 | 3.94E−07 | 6.7 | 4.4 | 10.6 | 0 | 146 | 854 |
| 20 | 14 | 19.5 | 1.98E−09 | 5.0 | 5.54E−05 | 6.7 | 4.4 | 11.0 | 0 | 166 | 834 |
| 20 | 238 | 5.2 | 4.89E−05 | 10.6 | 1.18E−07 | 6.7 | 4.4 | 10.9 | 0 | 168 | 832 |
| 20 | 217 | 13.1 | 8.99E−09 | 5.6 | 3.78E−05 | 6.7 | 4.5 | 10.2 | 0 | 135 | 865 |
| 20 | 715 | 10.7 | 1.06E−06 | 5.1 | 7.30E−06 | 6.6 | 4.3 | 10.4 | 0 | 158 | 842 |
| 20 | 336 | 9.6 | 5.66E−06 | 6.7 | 8.54E−06 | 6.4 | 4.2 | 9.8 | 0 | 238 | 762 |
| 20 | 505 | 13.7 | 2.92E−08 | 5.3 | 6.74E−06 | 6.3 | 4.1 | 9.9 | 0 | 258 | 742 |
| 20 | 905 | 5.5 | 8.14E−06 | 9.5 | 1.31E−07 | 6.2 | 4.1 | 9.8 | 0 | 279 | 721 |
| 20 | 761 | 10.7 | 6.27E−09 | 5.1 | 5.39E−05 | 6.2 | 4.0 | 9.9 | 0 | 267 | 733 |
| 20 | 631 | 10.8 | 1.23E−06 | 5.1 | 1.05E−05 | 6.2 | 3.9 | 9.9 | 0 | 278 | 722 |
| 20 | 169 | 8.5 | 3.47E−07 | 6.5 | 0.000127 | 6.2 | 4.1 | 10.0 | 0 | 242 | 758 |
| 20 | 763 | 8.5 | 8.51E−07 | 7.2 | 2.02E−06 | 6.1 | 4.1 | 9.8 | 0 | 294 | 706 |
| 20 | 82 | 9.8 | 5.66E−08 | 5.3 | 3.62E−05 | 6.1 | 4.0 | 9.6 | 0 | 278 | 722 |
| 20 | 764 | 5.6 | 6.00E−06 | 9.5 | 9.34E−08 | 6.1 | 3.9 | 9.5 | 0 | 277 | 723 |
| 20 | 608 | 7.5 | 1.13E−07 | 7.8 | 3.64E−05 | 6.0 | 4.0 | 9.3 | 0 | 264 | 736 |
| 20 | 330 | 17.0 | 1.29E−08 | 6.0 | 3.02E−07 | 5.9 | 4.0 | 9.0 | 0 | 293 | 707 |
| 20 | 951 | 10.6 | 3.00E−07 | 5.3 | 0.000206 | 5.8 | 3.8 | 9.2 | 0 | 349 | 651 |
| 20 | 647 | 11.8 | 2.24E−07 | 6.4 | 0.00017 | 5.8 | 3.9 | 8.9 | 0 | 328 | 672 |
| 20 | 226 | 12.0 | 3.32E−09 | 5.1 | 7.51E−05 | 5.8 | 3.7 | 9.0 | 0 | 383 | 617 |
| 20 | 122 | 5.3 | 1.98E−05 | 19.6 | 2.06E−06 | 5.5 | 3.5 | 8.6 | 0 | 446 | 554 |
| 20 | 216 | 7.9 | 7.20E−06 | 9.2 | 2.16E−09 | 5.4 | 3.4 | 8.8 | 0 | 511 | 489 |
| 20 | 680 | 10.4 | 1.11E−06 | 5.2 | 4.21E−06 | 5.3 | 3.6 | 8.1 | 0 | 532 | 468 |

3. Overall Survival and Response to Primary Therapy in SCCC

Cervical cancer remains a major contributor to female mortality worldwide (Cohen et al. 2019). For women diagnosed with locally advanced disease the cornerstone of treatment remains a combination radiation, chemotherapy, and surgery (Landoni et al. 1997) (Rose et al. 1999). However, therapeutic decisions remain multifactorial, based on stage, pathologic variables, and treatment side effects. Unfortunately, none of the prior are predictive of treatment response or molecular alterations which could be targeted to extend survival and treatment recommendations (Sedlis et al. 1999) (Peters III et al. 2000).

For these reasons the score presented herein has the potential to greatly impact care of cervical cancer patients. First, the genetic score was able to identify patients who have an excellent prognosis whether they received radiation or not. Even early-stage patients in the low-risk group with poor pathologic findings such as LVSI or advanced stage had 5-year overall survival exceeding 80%. This may allow for patients with low-risk scores to be triaged to undergoing the treatment that will result in the greatest quality of life for them in the long term. Furthermore, with additional studies this work may serve to replace Sedlis or Peter's criteria (Sedlis et al. 1999, Peters III et al. 2000). Second, those in the high-risk group demonstrated a phenotype that was remarkably resistant to treatment, as a combination of radiation and chemotherapy did not improve survival among this subgroup of patients. This represents a group of patients who would benefit from novel therapies, early initiation of palliative care, or both.

When examining functions of the 20 genes making up the TGS/RRS score, the three main pathways observed were related to hypoxia survival, which would contribute to radiation resistance, DNA repair activation which would confer resistance to both chemotherapy and radiation, and immune evasion. PFKP, PLOD1, QSOX1, LIF, ANGPTL4, and GRB10 were all associated with surviving hypoxic conditions or reducing reactive oxygen species, which would theoretically contribute to radiation resistance (Peng et al. 2019, Qi and Xu 2018, Coppock and Thorpe 2006, Liu et al. 2013, Metcalfe 2011, Kim et al. 2011, Holt and Siddle 2005). While MMS19, BRSK1, GALNT3, and LIF have roles in either nucleotide excision pathway, homologous recombination pathway, or responding to DNA damage (Kou et al. 2008, Chen and Vogel 2009, Sheta et al. 2019, Liu et al. 2013, Metcalfe 2011). Last, LIF, NRP1, and CD46 all have essential roles in anti-tumoral immunity including promoting Tregs, suppressing CD8+ T cells, and decreasing TH1 responses (Liu et al. 2013, Metcalfe 2011, Acharya and Anderson 2020, Cardone, Le Friec, and Kemper 2011).

Prior genetic risk scores in cervical cancer have been limited by the low number of patients or lack of association with treatment response (Wong et al. 2003) (Huang et al. 2012) (Lee et al. 2013). In another paper, which used the TCGA, Wang et al. were able to find a 9 gene combination which was predictive of survival, but did not expand on the proportion of early stage patients in each group, proportion with LVSI, or how patient score related to known pathologic risk factors such as LVSI (Wang et al. 2019). There was only 1 common gene between the Wang et al. score and the score presented herein, PEAR1 (platelet endothelial aggregation receptor 1). Comparing to the prior mentioned publications, the disclosure presented herein consisted of a large sample size, had improved survival prediction, and was associated with treatment response (Wong et al. 2003, Huang et al. 2012) (Lee et al. 2013, Wang et al. 2019).

The data represents an exciting advancement in cervical cancer. The score presented herein demonstrated excellent survival prediction along with biologically targetable mechanisms, which could be used to extend patient survival. However, future studies are needed to validate this risk score.

### EXAMPLES

### Example 1. Patient Characteristics

Materials and Methods:

Study Design and Patients:

Squamous cell cervical cancer patients from the TCGA patient cohort (n=203) were obtained through the UCSC Xena platform. All patients had level 3, log 2 transformed RNAseq data. Patients were divided into overarching stage groups (I, II, III, IV). FIGO stage breakdown for all patients can be found in Table 3. Overall survival was the primary endpoint of this disclosure.

Statistical Analyses:

Statistical analyses were performed using the R language and environment for statistical computing (Team 2013). Categorical variables were compared using Chi-squared test. Continuous variables were compared using student's t-test. P-values were considered significant if the value was less than 0.05. In single gene testing utilizing quartiles, the first quartile was used as the reference.

TABLE 3
FIGO staging of all patients

| | |
|---|---|
| | Patients # (%) |
| Characteristic | (n, total = 203) |

Stage

| | | |
|---|---|---|
| I | 3 | (2%) |
| IA1 | 1 | (<1%) |
| IB | 30 | (15%) |
| IB1 | 47 | (23%) |
| IB2 | 21 | (10%) |
| II | 3 | (2%) |
| IIA | 5 | (2%) |
| IIA1 | 5 | (2%) |
| IIA2 | 7 | (4%) |
| IIB | 30 | (15%) |
| III | 1 | (<1%) |
| IIIA | 2 | (1%) |
| IIIB | 29 | (14%) |
| IVA | 8 | (4%) |
| IVB | 6 | (4%) |
| Unknown | 5 | (2%) |

Results:

Of the 203 squamous cell cervical cancer patients identified the median age was 47 years old and 47% (n=96) were moderately differentiated tumors. Stage I disease made up 50% of the cohort. A total of 115 (57%) of patients had known lymph nodes assessment at initial diagnosis and 118 (58%) underwent adjuvant therapy. Demographic information is further summarized in Table 4. On univariate analysis stage IV disease, lymphovascular invasion, presence of positive lymph nodes, partial response to primary therapy, and no response to primary therapy were all associated with worse overall survival Table 4 and FIG. 5.

TABLE 4
Summary of demographic, pathologic, and treatment information for all patients.

| | | Patients # (%) | | Percent 5-year | | | |
|---|---|---|---|---|---|---|---|
| Characteristic | | (n, total = 203) | | Survival | HR(95% CI) | | p-value |
| Age | <47 years | 100 | (49%) | 67% | | | |
| | ≥47 years | 103 | (51%) | 65% | 1.22 | (0.73-2.04) | 0.44 |
| Stage* | 1 | 102 | (50%) | 68% | | | |
| | 2 | 50 | (25%) | 76% | 0.80 | (0.39-1.66) | 0.55 |
| | 3 | 32 | (16%) | 63% | 1.29 | (0.63-2.69) | 0.47 |
| | 4 | 14 | (7%) | 17% | 5.27 | (2.66-10.4) | <0.001 |
| | Unknown | 5 | (2%) | 100% | *Unable to | | 1 |
| | | | | | calculate | | |
| Histology | Squamous | 203 | (100%) | NA | NA | | NA |
| Non-Keratinizing | Non-Keratinizing | 87 | (43%) | 79% | | | |
| vs Keratinizing | Keratinizing | 43 | (21%) | 54% | 1.73 | (0.90-3.35) | 0.10 |
| | Unknown | 73 | (36%) | 65% | 1.25 | (0.66-2.34) | 0.49 |
| Grade | High | 77 | (38%) | 67% | | | |
| | Moderate | 96 | (47%) | 71% | 1.09 | (0.61-1.95) | 0.77 |
| | Low | 10 | (5%) | *Unable to | 1.01 | (0.24-4.37) | 0.99 |
| | | | | calculate | | | |
| | Unknown | 20 | (10%) | 34% | 2.50 | (1.16-5.39) | 0.02 |
| Lymphovascular | Absent | 43 | (21%) | 94% | | | |
| Invasion | Present | 55 | (27%) | 65% | 15.0 | (2.00-111.7) | 0.008 |
| | Unknown | 105 | (52%) | 57% | 16.8 | (2.31-122.4) | 0.005 |
| Positive Lymph | No | 77 | (38%) | 79% | | | |
| Nodes | Yes | 41 | (20%) | 64% | 2.19 | (1.04-4.61) | 0.04 |
| | Unknown | 85 | (42%) | 56% | 2.55 | (1.34-4.87) | 0.004 |
| Hysterectomy | Radical | 102 | (50%) | 75% | | | |
| Type Performed | Simple | 2 | (1%) | *Unable to | *Unable to | | 1 |
| | | | | calculate | calculate | | |
| | Unknown | 99 | (49%) | 57% | 1.83 | (1.09-3.08) | 0.02 |
| Treatment | None | 41 | (20%) | 64% | | | |
| | Radiation alone | 21 | (10%) | 61% | 1.16 | (0.42-3.19) | 0.78 |
| | Chemotherapy with | 97 | (48%) | 71% | 1.11 | (0.43-1.88) | 0.78 |
| | radiation | | | | | | |
| | Unknown | 44 | (22%) | 58% | 1.33 | (0.62-2.87) | 0.46 |
| Response to | Complete | 132 | (65%) | 80% | | | |
| Primary | Response | | | | | | |
| Treatment | Partial Response | 6 | (3%) | 0% | 7.72 | (2.59-23.1) | <0.001 |
| | Stable Disease | 4 | (2%) | *Unable to | *Unable to | | 1 |
| | | | | calculate | calculate | | |
| | No Response | 18 | (9%) | 0% | 18.2 | (9.17-36.2) | <0.001 |
| | Unknown | 43 | (21%) | 59%% | 2.91 | (1.54-5.52) | 0.001 |

*HR hazard ratio, CI confidence interval
*Table 1 for FIGO stage break down
*Unable to calculate secondary to follow up of less than 5 years in these patients or not enough events

### Example 2. Identification of Genes Associated with Poor Survival

Materials and Methods:

TCGA Cervical Squamous Cell Carcinoma (SCC) Dataset:

The RNAseq data (IlluminaHiSeq: log 2-normalized count+1) for SCCC from TCGA was downloaded from UCSC Xena (Goldman, et al., bioRxiv, 326470 (2019)). The details regarding the clinical characteristics of this dataset are available in a recent publication from TCGA (Cancer Genome Atlas Research Network, Nature, 543:378 (2017)). The TCGA dataset was used for this disclosure because it has the largest number of patients and the highest quality gene expression data of any publicly available dataset of patients with cervical cancer. Given the inherent molecular differences between the 2 histologic subtypes of cervical cancer, the analysis described herein was focused on SCC. The rationale was that SCC is the most common cervical cancer subtype and there were far more patient-derived samples for SCC than for adenocarcinoma in TCGA cervical cancer dataset. RNA-seq data for a total of 20,530 genes was available for each patient sample analyzed in this disclosure. Samples were included in this disclosure if they were SCCC and had both RNAseq and OS data available. Accordingly, samples were excluded from the disclosure if they (a) did not contain SCC, (b) contained SCC but were mixed with another histologic subtype (e.g., a mixed SCC and adenocarcinoma tumor), (c) did not contain RNA-seq data, or (d) did not contain OS data.

A total of 203 patients with SCCC met inclusion criteria for this analysis. Median age of the sampled population was 47 years. Median follow-up was 27.3 months. Stage distribution was as follows: I (102; 50.2%), II (50; 24.6%), III (32; 15.8%), IV (14; 6.9%), unknown (5; 2.5%). As of last follow-up, 143 (70.4%) of patients were alive, and 60 (29.6%) had died.

Statistical Analyses:

All statistical analyses were performed using the R language and environment for statistical computing (R version 3.2.2; R Foundation for Statistical Computing; www.r-project.org). The Cox proportional hazards models were used to evaluate the impact of gene expression levels on overall survival. Overall survival data (diagnosis to date of death) were downloaded from TCGA patient phenotype files. Patients who were alive were censored at the date of last follow-up visit. Kaplan-Meier survival analysis and log-rank test were used to compare differences in overall survival between groups classified using different cut-offs of expression level.

Identification of Survival-Associated Genes:

Survival differences associated with each gene were initially examined using 10 different cut-offs corresponding to each decile. For example, for the 90% cutoff, the top 10% of patients with the highest expression levels for a given gene were assigned to a “high expression” group and the bottom 90% of patients are assigned to a “low expression” group and the two groups of patients were analyzed using a univariate Cox regression analysis. Similarly, the top 80% of patients with the highest expression could also be compared to the remaining 20% of patients. For individual genes, the difference in survival for above and below the cut-off was assessed using hazard ratio (HR) and log-rank test, with a significance level of P<0.01. This process was repeated for each gene and at each cutoff.

Survival differences associated with each gene were also examined after dividing the patients into four quartiles based on gene expression levels for each gene. Survival for patients in the second, third and fourth quartile were compared to patients in the first quartile. Genes were ranked based on hazard ratio (HR) and log-rank test. These procedures allowed identification of genes that had large survival differences and could consistently predict survival at different cutoffs.

To accomplish these goals, survival analysis was systematically conducted for every gene and at every decile cutoff. Examination of the results suggested that larger survival differences were usually observed at the fourth quartile, although survival differences were also seen at the third and sometimes the second quartile.

Results:

Using selection criteria, 40 genes had good survival prediction potential as shown by the HR and p values (Table 5) and the Kaplan-Meier survival curves for representative genes (FIG. 1). These genes individually have good prognostic value. The functions of 40 high-risk genes were evaluated by pathway analysis supplemented by manual curation. Fifteen of the 40 genes (ANGPTL4, FNDC3A, GALNT2, GALNT3, GLG1, KBTBD6, LAMC1, LIF, MMS19, MTDH, NRP1, PFKP, PLOD1, QSOX1, ZNF281) are implicated in metastasis, migration and/or invasion; 11 genes (ANGPTL4, APCDD1L, COPA, FNDC3A, GALNT3, KBTBD6, LIF, MTDH, NRP1, PLAGL1, RPS6KA2) in cell proliferation; 4 genes (CD46, CD59, KBTBD2, NRP1) in immune suppression; and 3 genes (GRB10, NRP1, PEAR1) in angiogenesis. The functions of the genes are consistent with their association with poor survival as observed in this disclosure.

TABLE 5
Performance of the top 40 genes compared by quartile and continuous expression values

| | | contin- | continuous | contin- | | | | | | | |
|---|---|---|---|---|---|---|---|---|---|---|---|
| Order | gene | uous.HR | p.value | uous.concordance | Q2.HR | Q3.HR | Q4.HR | Q2 p | Q3 p | Q4 p | Overall p |
| 1 | EGLN1 | 2.76 | 0.0000 | 0.71 | 1.23 | 1.72 | 4.33 | 0.64 | 0.19 | 0.0001 | 0.0001 |
| 2 | CD46 | 2.83 | 0.0000 | 0.61 | 1.65 | 1.76 | 3.61 | 0.25 | 0.19 | 0.0011 | 0.0049 |
| 3 | PLOD1 | 1.89 | 0.0001 | 0.67 | 1.97 | 2.11 | 3.53 | 0.11 | 0.08 | 0.0015 | 0.0102 |
| 4 | QSOX1 | 1.87 | 0.0004 | 0.63 | 2.75 | 1.12 | 3.50 | 0.01 | 0.81 | 0.0016 | 0.0008 |
| 5 | TM2D1 | 2.08 | 0.0000 | 0.61 | 3.56 | 3.95 | 4.73 | 0.01 | 0.01 | 0.0019 | 0.0031 |
| 6 | PEAR1 | 1.50 | 0.0000 | 0.67 | 1.93 | 2.77 | 3.66 | 0.14 | 0.02 | 0.0021 | 0.0074 |
| 7 | FKBP9 | 2.15 | 0.0001 | 0.67 | 1.67 | 1.58 | 3.17 | 0.23 | 0.29 | 0.0032 | 0.0145 |
| 8 | NRP1 | 1.62 | 0.0003 | 0.63 | 1.32 | 1.70 | 2.95 | 0.51 | 0.21 | 0.0041 | 0.0160 |
| 9 | GALNT2 | 1.77 | 0.0000 | 0.68 | 0.90 | 1.61 | 2.93 | 0.80 | 0.22 | 0.0043 | 0.0043 |
| 10 | TMED4 | 2.85 | 0.0002 | 0.66 | 1.93 | 1.45 | 3.04 | 0.11 | 0.41 | 0.0046 | 0.0190 |
| 11 | KIRREL | 1.22 | 0.0021 | 0.65 | 1.46 | 1.65 | 2.69 | 0.36 | 0.22 | 0.0075 | 0.0470 |
| 12 | LAMC1 | 1.69 | 0.0003 | 0.65 | 1.52 | 1.86 | 2.83 | 0.33 | 0.14 | 0.0083 | 0.0412 |
| 13 | SDF4 | 2.66 | 0.0011 | 0.60 | 1.06 | 1.31 | 2.61 | 0.89 | 0.50 | 0.0089 | 0.0256 |
| 14 | COPA | 2.55 | 0.0027 | 0.60 | 0.84 | 0.65 | 2.44 | 0.65 | 0.30 | 0.0098 | 0.0015 |
| 15 | FNDC3A | 1.99 | 0.0003 | 0.62 | 1.27 | 2.23 | 2.76 | 0.58 | 0.04 | 0.0105 | 0.0249 |
| 16 | GALNT3 | 1.64 | 0.0037 | 0.61 | 1.12 | 1.07 | 2.50 | 0.78 | 0.86 | 0.0136 | 0.0290 |
| 17 | PLK1S1 | 1.70 | 0.0032 | 0.60 | 1.42 | 1.62 | 2.57 | 0.41 | 0.26 | 0.0169 | 0.0794 |
| 18 | ANGPTL4 | 1.25 | 0.0088 | 0.60 | 1.29 | 1.24 | 2.46 | 0.53 | 0.59 | 0.0181 | 0.0831 |
| 19 | APCDD1L | 1.21 | 0.0008 | 0.65 | 1.78 | 1.77 | 2.37 | 0.16 | 0.14 | 0.0229 | 0.1320 |
| 20 | ZNF281 | 1.40 | 0.0043 | 0.65 | 1.06 | 1.88 | 2.38 | 0.89 | 0.11 | 0.0241 | 0.0435 |
| 21 | MMS19 | 1.93 | 0.0292 | 0.62 | 0.77 | 0.88 | 2.09 | 0.50 | 0.74 | 0.0307 | 0.0246 |
| 22 | GPR27 | 1.23 | 0.0146 | 0.59 | 1.63 | 1.77 | 2.23 | 0.22 | 0.15 | 0.0366 | 0.1903 |
| 23 | MTDH | 2.01 | 0.0229 | 0.57 | 2.34 | 1.97 | 2.35 | 0.04 | 0.10 | 0.0389 | 0.1101 |
| 24 | LIF | 1.35 | 0.0008 | 0.62 | 1.48 | 1.94 | 2.24 | 0.33 | 0.10 | 0.0412 | 0.1689 |
| 25 | BRSK1 | 1.22 | 0.0109 | 0.59 | 1.99 | 1.36 | 2.19 | 0.08 | 0.47 | 0.0448 | 0.1413 |
| 26 | GLG1 | 2.24 | 0.0091 | 0.63 | 0.36 | 0.73 | 1.87 | 0.03 | 0.42 | 0.0449 | 0.0004 |
| 27 | KBTBD2 | 2.24 | 0.0347 | 0.58 | 0.66 | 0.84 | 1.96 | 0.33 | 0.65 | 0.0459 | 0.0141 |
| 28 | PFKP | 1.61 | 0.0059 | 0.65 | 0.89 | 1.34 | 2.01 | 0.77 | 0.46 | 0.0523 | 0.0893 |
| 29 | CD59 | 1.41 | 0.0266 | 0.58 | 0.53 | 0.69 | 1.85 | 0.12 | 0.31 | 0.0659 | 0.0073 |
| 30 | PLAGL1 | 1.20 | 0.0278 | 0.59 | 1.18 | 1.25 | 1.92 | 0.67 | 0.56 | 0.0778 | 0.3117 |
| 31 | PRR12 | 1.75 | 0.0237 | 0.58 | 1.57 | 1.15 | 1.85 | 0.24 | 0.75 | 0.1012 | 0.3203 |
| 32 | KBTBD6 | 1.45 | 0.0998 | 0.56 | 1.23 | 0.72 | 1.73 | 0.57 | 0.44 | 0.1106 | 0.1315 |
| 33 | GRB10 | 1.31 | 0.0180 | 0.61 | 1.24 | 1.23 | 1.78 | 0.58 | 0.60 | 0.1146 | 0.4310 |
| 34 | ZC3H12C | 1.27 | 0.0616 | 0.55 | 1.50 | 1.14 | 1.66 | 0.30 | 0.74 | 0.1843 | 0.4971 |
| 35 | FSD1L | 1.30 | 0.0339 | 0.58 | 1.04 | 0.75 | 1.57 | 0.93 | 0.47 | 0.1971 | 0.2382 |
| 36 | AIMP2 | 1.72 | 0.0229 | 0.62 | 1.32 | 1.40 | 1.63 | 0.47 | 0.37 | 0.2033 | 0.6318 |
| 37 | ZNF701 | 1.25 | 0.0280 | 0.57 | 0.67 | 0.94 | 1.54 | 0.33 | 0.87 | 0.2134 | 0.1596 |
| 38 | RPS6KA2 | 1.19 | 0.1063 | 0.59 | 1.13 | 0.87 | 1.52 | 0.73 | 0.73 | 0.2256 | 0.4869 |
| 39 | TMEM167A | 1.67 | 0.1151 | 0.56 | 0.74 | 0.74 | 1.49 | 0.42 | 0.45 | 0.2269 | 0.1547 |
| 40 | RNF145 | 1.26 | 0.2585 | 0.51 | 0.62 | 0.79 | 1.21 | 0.22 | 0.51 | 0.5771 | 0.3183 |

### Example 3. Transcriptomic Risk Score (TRS) Using Machine Learning

Materials and Methods:

Building the SCCC Gene Signature and TRS Stratifier:

The individual genes with high survival differences were used to construct a survival prediction model using a machine learning method. The least absolute shrinkage selection operator (LASSO) algorithm was used to select and fit the regression coefficients for each gene in a penalized Cox proportional hazard model (Simon, et al., J Stat Softw. 39:1(2011); Friedman, et al., J Stat Softw. 33:1 (2010)). This process allowed us to select a subset of the genes, with weighted expression values, to use in calculating a survival risk score for each patient. The risk scores were then used to stratify all patients into 3 transcriptomic risk score (TRS) or Ridge regression score (RRS) groups. The stratification was optimized using the log-rank test. For the univariate analysis, major clinical characteristics with prognostic relevance were fitted to a Cox regression model after removal of patients with unknown clinical information. All clinical variables that were significant on univariate analysis (stage and lymph node status) were combined with TRS for the multivariate Cox model. Although LASSO is capable of selecting genes, it is not possible to apply LASSO to the entire genomic dataset with over 20,000 genes and come up with the best model. Therefore, the approach presented herein of pre-selecting genes using unigene survival analyses and then fitting a LASSO model represents a practical and efficient way of developing multivariate models.

Validation:

Because there was no hold out validation set, bootstrapping was performed to validate models. The mean HR was computed for 1,000 bootstraps utilizing 70% of the data set in each bootstrap. A model was considered valid if 95% or more of the bootstrapped models had a p-value 0.05 or less.

Results:

The 40 genes identified using the described selection criteria were used to identify the gene signatures that can predict survival. Ridge regression was then performed utilizing the “glmnet” package in R in order to make a prognostic score combining all genes (Friedman, Hastie, and Tibshirani 2009). With the 40 genes, Ridge regression analysis was conducted to find the optimal regression coefficients and decipher which composition of genes was most predictive of survival. This was performed in 3,000 training and testing pairs. Of the 3,000 models, 86 had an HR greater than 5 in both the training and test set. Based on individual gene RRS, it was determined that 25 of the 40 genes contributed the majority of the points to each individual risk score. Therefore, the top 25 genes were selected and shown in Table 1 and Table 5.

Using the 25 genes with the highest RRS scores, the same process was repeated which resulted in 190 models with a hazard ratio of greater than 5 in both the training and test sets. A final iteration was performed using the top 20/25 genes. This resulted in 245 models with an HR of greater than 5 in both training and test sets. The best 40 models for both the 25 and 20 gene signatures are shown in Table 6. These models were further evaluated by bootstrapping for 1,000 bootstraps over the entire dataset which showed that each model retained its prognostic capabilities as shown in Table 6. Almost all models had close to a 40% difference in percent 5-year overall survival, representative models shown in FIG. 2.

TABLE 6
Top Ridge regression models and their performances for survival prediction.

| | | Training testing 50% split | | | | Bootstrapping | | | | |
|---|---|---|---|---|---|---|---|---|---|---|
| Gene | Model | HR: | p-value | HR: | p-value | HR | | p > | p: 0.05 | p < |
| Set | # | train | train | test | test | (95% CI) | | 0.05 | to E−06 | E−6 |
| 25 | 910 | 5.31 | 6.84E−05 | 9.51 | 2.75E−08 | 8.51 | (5.1-15.5) | 0 | 32 | 968 |
| 25 | 70 | 6.22 | 1.84E−05 | 8.49 | 7.38E−07 | 8.23 | (5.15-14.81) | 0 | 43 | 957 |
| 25 | 473 | 13.5 | 6.57E−09 | 5.01 | 9.42E−05 | 8.18 | (5.26-13.86) | 0 | 33 | 967 |
| 25 | 835 | 8.13 | 4.03E−07 | 7.27 | 3.37E−06 | 8 | (5.12-13.16) | 0 | 46 | 954 |
| 25 | 789 | 12.3 | 2.30E−09 | 5.11 | 6.05E−05 | 7.96 | (5.07-13.63) | 0 | 44 | 956 |
| 25 | 931 | 8.43 | 6.88E−07 | 6.8 | 1.23E−05 | 7.79 | (5.11-12.57) | 0 | 41 | 959 |
| 25 | 1000 | 9.48 | 4.02E−06 | 6.32 | 1.17E−06 | 7.78 | (4.97-12.37) | 0 | 53 | 947 |
| 25 | 630 | 8.43 | 2.81E−07 | 6.33 | 5.15E−06 | 7.62 | (5.02-12.23) | 0 | 65 | 935 |
| 25 | 614 | 5.27 | 5.22E−06 | 10.6 | 3.34E−07 | 7.53 | (4.92-11.76) | 0 | 65 | 935 |
| 25 | 193 | 5.53 | 3.66E−05 | 9.22 | 3.83E−07 | 7.44 | (4.82-12.41) | 0 | 80 | 920 |
| 25 | 544 | 5.91 | 1.09E−05 | 8.92 | 3.85E−06 | 7.31 | (4.65-11.69) | 0 | 78 | 922 |
| 25 | 351 | 5.68 | 2.11E−05 | 10.8 | 3.33E−07 | 7.3 | (4.84-12.51) | 0 | 96 | 904 |
| 25 | 897 | 7.39 | 1.35E−06 | 8.17 | 6.34E−07 | 7.29 | (4.56-12.6) | 0 | 105 | 895 |
| 25 | 447 | 9.65 | 4.72E−07 | 5.76 | 1.08E−05 | 7.26 | (4.49-12.26) | 0 | 115 | 885 |
| 25 | 964 | 11 | 8.51E−08 | 5.59 | 1.61E−05 | 7.19 | (4.6-12.19) | 0 | 94 | 906 |
| 25 | 176 | 6.64 | 1.29E−06 | 8.67 | 3.06E−07 | 6.97 | (4.67-10.82) | 0 | 123 | 877 |
| 25 | 529 | 9.73 | 1.73E−06 | 5.48 | 1.06E−05 | 6.92 | (4.53-11.09) | 0 | 140 | 860 |
| 25 | 383 | 10.9 | 6.21E−08 | 5.51 | 4.30E−05 | 6.9 | (4.26-11.74) | 0 | 144 | 856 |
| 25 | 264 | 6.25 | 4.14E−06 | 9.55 | 1.56E−06 | 6.88 | (4.45-10.73) | 0 | 99 | 901 |
| 25 | 434 | 6.19 | 4.54E−07 | 10.2 | 8.79E−07 | 6.83 | (4.39-11.26) | 0 | 148 | 852 |
| 25 | 605 | 6.47 | 3.07E−06 | 10.1 | 7.68E−07 | 6.81 | (4.34-11.3) | 0 | 152 | 848 |
| 25 | 950 | 10.6 | 3.70E−08 | 6.79 | 1.69E−06 | 6.8 | (4.23-11.16) | 0 | 150 | 850 |
| 25 | 21 | 10.3 | 7.03E−07 | 5.36 | 8.18E−06 | 6.77 | (4.25-11.1) | 0 | 163 | 837 |
| 25 | 115 | 9.4 | 1.24E−07 | 5.47 | 6.12E−05 | 6.69 | (4.34-10.43) | 0 | 150 | 850 |
| 25 | 246 | 8.98 | 1.14E−07 | 6.45 | 2.86E−05 | 6.69 | (4.26-10.52) | 0 | 179 | 821 |
| 25 | 172 | 8.6 | 7.18E−07 | 6.74 | 8.08E−07 | 6.66 | (4.32-10.81) | 0 | 174 | 826 |
| 25 | 202 | 9.72 | 3.04E−06 | 6.03 | 9.69E−07 | 6.61 | (4.34-10.96) | 0 | 168 | 832 |
| 25 | 607 | 9.33 | 2.76E−07 | 6.62 | 1.46E−06 | 6.5 | (4.01-10.68) | 0 | 247 | 753 |
| 25 | 27 | 9.36 | 5.39E−06 | 5.76 | 4.19E−06 | 6.46 | (4.33-10.36) | 0 | 204 | 796 |
| 25 | 763 | 9.36 | 2.56E−07 | 6.14 | 1.41E−05 | 6.3 | (3.95-10.43) | 0 | 297 | 703 |
| 25 | 647 | 12.2 | 1.66E−07 | 8.24 | 1.20E−05 | 6.26 | (4.12-9.71) | 0 | 214 | 786 |
| 25 | 710 | 5.64 | 2.49E−05 | 11.2 | 3.32E−07 | 6.22 | (4.19-9.71) | 0 | 217 | 783 |
| 25 | 999 | 9.22 | 1.07E−06 | 6.08 | 1.63E−05 | 6.11 | (4.06-9.65) | 0 | 265 | 735 |
| 25 | 255 | 9.63 | 3.16E−07 | 5.16 | 1.64E−05 | 6.11 | (3.83-10.14) | 0 | 331 | 669 |
| 25 | 82 | 15.4 | 5.95E−10 | 5.74 | 1.47E−05 | 6.08 | (3.85-10.49) | 0 | 331 | 669 |
| 25 | 761 | 9.9 | 1.56E−08 | 5.22 | 3.74E−05 | 5.99 | (3.83-9.74) | 0 | 319 | 681 |
| 25 | 764 | 6.8 | 6.73E−07 | 8.11 | 7.82E−07 | 5.95 | (3.93-9.52) | 0 | 334 | 666 |
| 25 | 656 | 10.6 | 1.59E−06 | 5.01 | 4.69E−05 | 5.77 | (3.86-8.84) | 0 | 375 | 625 |
| 25 | 330 | 12.1 | 1.29E−07 | 5.97 | 3.02E−07 | 5.77 | (3.93-9.09) | 0 | 379 | 621 |
| 25 | 799 | 5.42 | 1.71E−05 | 9.41 | 7.70E−08 | 5.77 | (3.61-9.35) | 0 | 431 | 569 |
| 20 | 632 | 10 | 2.64E−07 | 10.5 | 2.71E−07 | 9.6 | (5.9-16.8) | 0 | 6 | 994 |
| 20 | 70 | 8.8 | 1.24E−06 | 7.2 | 5.26E−07 | 8.6 | (4.6-15.8) | 0 | 52 | 948 |
| 20 | 793 | 15.3 | 5.25E−10 | 5.3 | 0.000288 | 8.3 | (5.3-14) | 0 | 25 | 975 |
| 20 | 351 | 5.8 | 2.03E−05 | 10.4 | 7.87E−07 | 8.1 | (5.1-14.3) | 0 | 51 | 949 |
| 20 | 277 | 9.8 | 2.04E−07 | 6.2 | 1.11E−05 | 8 | (5.2-13.2) | 0 | 37 | 963 |
| 20 | 208 | 9.5 | 1.50E−07 | 6.9 | 1.31E−06 | 7.9 | (5.1-13.3) | 0 | 47 | 953 |
| 20 | 630 | 8.4 | 2.81E−07 | 6.9 | 1.18E−06 | 7.6 | (4.9-12.7) | 0 | 55 | 945 |
| 20 | 838 | 13.2 | 5.01E−08 | 5.5 | 3.83E−06 | 7.5 | (5.1-12.3) | 0 | 55 | 945 |
| 20 | 245 | 7.3 | 8.12E−07 | 9.1 | 6.07E−05 | 7.4 | (4.9-11.7) | 0 | 52 | 948 |
| 20 | 434 | 7.2 | 5.85E−08 | 10.3 | 4.07E−06 | 7.3 | (4.8-12) | 0 | 68 | 932 |
| 20 | 57 | 9.6 | 8.87E−08 | 6.2 | 1.15E−05 | 7.3 | (4.6-11.6) | 0 | 112 | 888 |
| 20 | 532 | 9 | 1.46E−06 | 6.9 | 6.67E−07 | 7.3 | (4.7-11.7) | 0 | 57 | 943 |
| 20 | 173 | 8.6 | 9.92E−07 | 6.6 | 1.01E−05 | 7.2 | (4.5-11.9) | 0 | 93 | 907 |
| 20 | 728 | 11.1 | 7.72E−09 | 5.6 | 5.06E−05 | 7.2 | (4.5-11.9) | 0 | 113 | 887 |
| 20 | 952 | 8.8 | 7.84E−08 | 6.2 | 6.17E−05 | 7.2 | (4.3-11.8) | 0 | 118 | 882 |
| 20 | 341 | 10 | 1.24E−06 | 5.4 | 1.10E−05 | 7 | (4.5-11.5) | 0 | 159 | 841 |
| 20 | 875 | 11.4 | 2.35E−07 | 5.6 | 5.11E−06 | 7 | (4.6-11.4) | 0 | 136 | 864 |
| 20 | 851 | 8.8 | 7.62E−07 | 17.4 | 7.12E−06 | 6.8 | (4.4-11) | 0 | 153 | 847 |
| 20 | 710 | 5.1 | 6.18E−05 | 11.3 | 3.94E−07 | 6.7 | (4.4-10.6) | 0 | 146 | 854 |
| 20 | 14 | 19.5 | 1.98E−09 | 5 | 5.54E−05 | 6.7 | (4.4-11) | 0 | 166 | 834 |
| 20 | 238 | 5.2 | 4.89E−05 | 10.6 | 1.18E−07 | 6.7 | (4.4-10.9) | 0 | 168 | 832 |
| 20 | 217 | 13.1 | 8.99E−09 | 5.6 | 3.78E−05 | 6.7 | (4.5-10.2) | 0 | 135 | 865 |
| 20 | 715 | 10.7 | 1.06E−06 | 5.1 | 7.30E−06 | 6.6 | (4.3-10.4) | 0 | 158 | 842 |
| 20 | 336 | 9.6 | 5.66E−06 | 6.7 | 8.54E−06 | 6.4 | (4.2-9.8) | 0 | 238 | 762 |
| 20 | 505 | 13.7 | 2.92E−08 | 5.3 | 6.74E−06 | 6.3 | (4.1-9.9) | 0 | 258 | 742 |
| 20 | 905 | 5.5 | 8.14E−06 | 9.5 | 1.31E−07 | 6.2 | (4.1-9.8) | 0 | 279 | 721 |
| 20 | 761 | 10.7 | 6.27E−09 | 5.1 | 5.39E−05 | 6.2 | (4-9.9) | 0 | 267 | 733 |
| 20 | 631 | 10.8 | 1.23E−06 | 5.1 | 1.05E−05 | 6.2 | (3.9-9.9) | 0 | 278 | 722 |
| 20 | 169 | 8.5 | 3.47E−07 | 6.5 | 0.000127 | 6.2 | (4.1-10) | 0 | 242 | 758 |
| 20 | 763 | 8.5 | 8.51E−07 | 7.2 | 2.02E−06 | 6.1 | (4.1-9.8) | 0 | 294 | 706 |
| 20 | 82 | 9.8 | 5.66E−08 | 5.3 | 3.62E−05 | 6.1 | (4-9.6) | 0 | 278 | 722 |
| 20 | 764 | 5.6 | 6.00E−06 | 9.5 | 9.34E−08 | 6.1 | (3.9-9.5) | 0 | 277 | 723 |
| 20 | 608 | 7.5 | 1.13E−07 | 7.8 | 3.64E−05 | 6 | (4-9.3) | 0 | 264 | 736 |
| 20 | 330 | 17 | 1.29E−08 | 6 | 3.02E−07 | 5.9 | (4-9) | 0 | 293 | 707 |
| 20 | 951 | 10.6 | 3.00E−07 | 5.3 | 0.000206 | 5.8 | (3.8-9.2) | 0 | 349 | 651 |
| 20 | 647 | 11.8 | 2.24E−07 | 6.4 | 0.00017 | 5.8 | (3.9-8.9) | 0 | 328 | 672 |
| 20 | 226 | 12 | 3.32E−09 | 5.1 | 7.51E−05 | 5.8 | (3.7-9) | 0 | 383 | 617 |
| 20 | 122 | 5.3 | 1.98E−05 | 19.6 | 2.06E−06 | 5.5 | (3.5-8.6) | 0 | 446 | 554 |
| 20 | 216 | 7.9 | 7.20E−06 | 9.2 | 2.16E−09 | 5.4 | (3.4-8.8) | 0 | 511 | 489 |
| 20 | 680 | 10.4 | 1.11E−06 | 5.2 | 4.21E−06 | 5.3 | (3.6-8.1) | 0 | 532 | 468 |

### Example 4. Consensus Modeling

Methods:

Consensus Voting:

Once the final model had been chosen and bootstrapping was completed validating the models, individual model prediction was combined utilizing consensus voting.

Results:

Because it is not expected that all models would rank patients into the same high and low risk groupings, the disclosure examined how often the 80 models with the highest mean HR (after bootstrapping) in both the 25 and 20 gene combinations agreed on patient risk grouping. If models concurred on patient risk grouping at least 75% of the time, then the patient was classified as that risk grouping. When models concurred less than 75% of the time, patients were classified as ambiguous or moderate risk. The 80 models agreed on placing 86/203 (42%) patients into the low risk group, and 83/203 (41%) patients into the high-risk group. For the remaining 34/203 (17%) patients, there was agreement less than 75% of the time, and therefore these patients were defined as a middle or ambiguous risk group (FIG. 4A). Patients in the high-risk group had a 58% decrease in percent 5-year overall survival (5-year overall survival is 90% for low risk and 32% for high risk, HR=11.1, 95% CI=5.18-23.7, p=5.99E-10). The moderate risk group had similar survival to the low-risk group (FIG. 4A). Given the number of clinical prognostic factors, covariate analysis was performed between the risk score and all significant clinical parameters. Covariable analysis showed that high risk score, presence of lymphovascular invasion, and no response to primary therapy were all associated with worse prognosis (Table 4).

### Example 5. Risk Score Subgroup Analysis

Results:

Surprisingly, 22% (18/86) of patients in the low risk group had stage III disease or greater, 31% (27/86) had positive LVSI, and 23% (20/86) had positive lymph node metastases (Table 7). Because of the large number of patients in the low-risk group with poor prognostic pathologic characteristics, a subgroup analysis was performed to see if TGS/RRS outweighed the potential negative effects of their pathologic characteristics. Patients in the low-risk group with unknown or absent LVSI had a percent 5-year overall survival of 87% compared to 34% among high-risk patients with positive LVSI (HR 10.3, 95% CI 4.01-26.7, p<0.001). Stage 3 and 4 patients in the low-risk group had a percent 5-year survival of 83%. Based on this, it is evident that the TGS/RRS outweighs the potential negative implications of poor pathologic findings. Importantly, the only clinical, pathologic, or treatment characteristics that had more frequent occurrence in the high-risk group were advanced stage (p=0.04) and worse response to primary therapy (p<0.001). There was no discrepancy between the receipt of radiation (p=0.89), chemotherapy (p=0.33), or surgery (p=0.33) between and high, moderate, or low risk group patients (Table 7).

TABLE 7
Summary of demographic, pathologic, and treatment information
comparing low, moderate, and high risk patients

| | | Low risk | | Moderate Risk | | High risk | | |
|---|---|---|---|---|---|---|---|---|
| Characteristic | | (n = 86) | | (n = 34) | | (n = 83) | | p-value |
| Age (median) | | 47 | | 44 | | 47 | | 0.81 |
| Stage | 1 | 45 | (52%) | 18 | (53%) | 39 | (47%) | 0.04 |
| | 2 | 20 | (23%) | 11 | (32%) | 19 | (23%) | |
| | 3 | 17 | (20%) | 3 | (9%) | 12 | (14%) | |
| | 4 | 1 | (2%) | 1 | (3%) | 12 | (14%) | |
| | Unknown | 3 | (3%) | 1 | (3%) | 1 | (1%) | |
| Non- | Non- | 40 | (47%) | 14 | (41%) | 33 | (40%) | 0.66 |
| Keratinizing vs | Keratinizing | | | | | | | |
| Keratinizing | Keratinizing | 18 | (21%) | 5 | (15%) | 20 | (24%) | |
| | Unknown | 28 | (33%) | 15 | (44%) | 30 | (36%) | |
| Grade | High | 29 | (34%) | 13 | (38%) | 35 | (42%) | 0.31 |
| | Moderate | 46 | (53%) | 17 | (50%) | 33 | (49%) | |
| | Low | 6 | (7%) | 1 | (3%) | 3 | (4%) | |
| | Unknown | 5 | (6%) | 3 | (9%) | 12 | (15%) | |
| Lymphovascular | Absent | 21 | (24%) | 8 | (24%) | 14 | (17%) | 0.4 |
| Invasion | Present | 27 | (31%) | 8 | (24%) | 20 | (24%) | |
| | Unknown | 38 | (44%) | 18 | (52%) | 49 | (59%) | |
| Positive Lymph | No | 40 | (47%) | 11 | (32%) | 26 | (31%) | 0.07 |
| Nodes | Yes | 20 | (23%) | 5 | (15%) | 16 | (19%) | |
| | Unknown | 26 | (30%) | 18 | (53%) | 41 | (49%) | |
| Hysterectomy | Radical | 47 | (55%) | 17 | (50%) | 38 | (46%) | 0.49 |
| Type Performed | Simple | 0 | (0%) | 1 | (3$) | 1 | (1%) | |
| | Unknown | 39 | (45%) | 16 | (47%) | 44 | (53%) | |
| Treatment | None | 18 | (21%) | 6 | (18%) | 17 | (20%) | 0.96 |
| | Radiation | 8 | (9%) | 5 | (15%) | 8 | (10%) | |
| | alone | | | | | | | |
| | Chemotherapy | 43 | (50%) | 16 | (47%) | 38 | (46%) | |
| | with radiation | | | | | | | |
| | Unknown | 17 | (20%) | 7 | (20%) | 20 | (24%) | |
| Response to | Complete | 65 | (76%) | 27 | (79%) | 40 | (48%) | <0.001 |
| Primary | Response | | | | | | | |
| Treatment | Partial | 0 | (0%) | 0 | (0%) | 6 | (8%) | |
| | Response | | | | | | | |
| | Stable Disease | 4 | (5%) | 0 | (0%) | 0 | (0%) | |
| | No Response | 1 | (1%) | 0 | (0%) | 17 | (20%) | |
| | Unknown | 16 | (19%) | 7 | (21%) | 20 | (24%) | |

Interestingly, 70% (n=57/83) of high-risk group patients had either stage I or II disease. When comparing only early stage low and high-risk patients, there was a persistent survival difference (5-year overall survival is 91% for low risk and 39% for high risk, HR=11.3, 95% CI=4.30-29.6, p=8.49E-07). Among stage I and II high-risk patients, there was no difference in survival regardless of whether patients received no treatment, radiation alone (HR=0.95, p=0.94) or if they received CRT (HR=1.19, p=0.67). This indicates high risk patients represent an extremely treatment-resistant population. Supporting this conclusion, there was a difference in response to primary therapy when comparing between low and high-risk patients (p<0.001). The high-risk group contained 94% (17/18) of patients who did not respond to primary therapy and 100% (6/6) of partial responders to primary therapy (Table 7).

### Example 6. Multivariate Analyses with TRS and Clinical Parameters

Results

Across the TRS groups, median age, stage, lymph node status, and grades were similarly represented (Table 7). Median overall survival was 1.56 years for the high-risk TRS group, 8.48 years for the intermediate-risk TRS group, and not yet reached for the low-risk TRS group.

Stage-by-stage distribution of the TRS groups can also be found in Table 7. Two observations from this part of the analysis are worth high-lighting. First, 40.9% of early stage patients (stage I and stage II) were in the TRS poor-survival subgroup. Second, 47.8% of late stage patients (stage 3 and 4) had low TRS, belonging to the good-survival TRS group, suggesting that TGS completely overwrites the contribution of stage.

Univariate analysis of major clinical variables for SCCC found that stage 4 and lymph node status were each significantly associated with survival, but grade was not (FIG. 5 and Table 7). Stage IV patients had very poor survival, while survival was not significantly different between stage I, II and III patients (FIG. 5A). On univariate analysis, the high-risk TRS group was 9-times more likely to die compared to the low-risk and moderate-risk TRS groups (HR=9.0, P<10E-15) (Table 8).

Given that stage was the most significant clinical factor associated with survival, survival analysis was further carried out on stage I-III patients stratified by TRS (FIGS. 4C and 4D). The TRS-stratified survival pattern for stage I-III patients was almost identical to that observed with the entire dataset with stage IV patients (FIGS. 4A and 4B), confirming that TRS-based survival differences were not confounded by stage. In addition, multivariate analysis using TRS as the dependent variable and clinical variables that were significant on univariate analysis as co-variables revealed high TRS as the most important survival predictor (HR=8.1; 95% CI=3.5-19.0; P<10E-5) (Table 8). In the multivariate analyses, both stage and lymphnode status do not contribute significantly to the survival risk (Table 8), suggesting that TGS is the only known risk factor for survival.

TABLE 8
Hazard ratios for TRS and major clinical factors.

| | Univariate analysis | | | Multivariate analysis | | |
|---|---|---|---|---|---|---|
| Characteristic | HR | 95% CI | P | HR | 95% CI | P |
| Transcriptomic risk | | | | | | |
| Low/M | | | | | | |
| High | 9.0 | 4.8 to 16.8 | <10E−15 | 8.1 | 3.5 to 19.0 | <10E−5 |
| Stage | | | | | | |
| I | Ref | | | | | |
| II | 0.66 | 0.28 to 1.52 | 0.33 | 0.9 | 0.5 to 1.7 | 0.8 |
| III | 1.23 | 0.58 to 2.63 | 0.59 | | | |
| IV | 6.22 | 2.89 to 13.4 | <0.001 | | | |
| Grade | | | | | | |
| 1 | | | | | | |
| 2 | 1.06 | 0.25 to 4.47 | 0.94 | | Not included | |
| 3 | 1.01 | 0.23 to 4.34 | 0.99 | | | |
| Lymph Node Status | | | | | | |
| Negative | | | | | | |
| Positive | 2.16 | 1.03 to 4.55 | 0.043 | 1.7 | 0.8 to 3.7 | 0.2 |

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

### REFERENCES

Acharya, Nandini, and Ana C Anderson. 2020. “NRP1 cripples immunological memory.” Nature Immunology 21 (9):972-973.
Cardone, J, G Le Friec, and C Kemper. 2011. “CD46 in innate and adaptive immunity: an update.” Clinical & Experimental Immunology 164 (3):301-311.
Chen, Daici, and Jackie Vogel. 2009. “SAD kinase keeps centrosomes lonely.” nature cell biology 11 (9):1047-1048.
Cohen, Paul A, Anjua Jhingran, Ana Oaknin, and Lynette Denny. 2019. “Cervical cancer.” The Lancet 393 (10167):169-182.
Coppock, Donald L, and Colin Thorpe. 2006. “Multidomain flavin-dependent sulfhydryl oxidases.” Antioxidants & redox signaling 8 (3-4):300-311.
Friedman, Jerome, Trevor Hastie, and Rob Tibshirani. 2009. “glmnet: Lasso and elastic-net regularized generalized linear models.” R package version 1 (4).
Holt, Lowenna J, and Kenneth Siddle. 2005. “Grb10 and Grb14: enigmatic regulators of insulin action—and more?” Biochemical Journal 388 (2):393-406.
Huang, Long, Min Zheng, Qing-Ming Zhou, Mei-Yin Zhang, Yan-Hong Yu, Jing-Ping Yun, and Hui-Yun Wang. 2012. “Identification of a 7-gene signature that predicts relapse and survival for early stage patients with cervical carcinoma.” Medical Oncology 29 (4):2911-2918.
Kim, S M, H S Choi, and J S Byun. 2000. “Overall 5-year survival rate and prognostic factors in patients with stage D3 and IIA cervical cancer treated by radical hysterectomy and pelvic lymph node dissection.” International Journal of Gynecological Cancer 10 (4):305-312.
Kim, Sun-Hee, Yun-Yong Park, Sang-Wook Kim, Ju-Seog Lee, Dingzhi Wang, and Raymond N DuBois. 2011. “ANGPTL4 induction by prostaglandin E2 under hypoxic conditions promotes colorectal cancer progression.” Cancer research 71 (22):7010-7020.
Kou, Haiping, Ying Zhou, R M Charlotte Gorospe, and Zhigang Wang. 2008. “Mms19 protein functions in nucleotide excision repair by sustaining an adequate cellular concentration of the TFIIH component Rad3.” Proceedings of the National Academy of Sciences 105 (41):15714-15719.
Landoni, Fabio, Alessandro Colombo, Rodolfo Milani, Franco Placa, Vanna Zanagnolo, and Costantino Mangioni. 2017. “Randomized study between radical surgery and radiotherapy for the treatment of stage IB-IIA cervical cancer: 20-year update.” Journal of gynecologic oncology 28 (3):e34-e34. doi: 10.3802/jgo.2017.28.e34.
Landoni, Fabio, Andrea Maneo, Alessandro Colombo, Franco Placa, Rodolfo Milani, Patrizia Perego, Giorgio Favini, Luigi Ferri, and Costantino Mangioni. 1997. “Randomised study of radical surgery versus radiotherapy for stage Ib-IIa cervical cancer.” The Lancet 350 (9077):535-540.
Lee, Yoo-Young, Tae-Joong Kim, Ji-Young Kim, Chel Hun Choi, In-Gu Do, Sang Yong Song, Insuk Sohn, Sin-Ho Jung, Duk-Soo Bae, and Jeong-Won Lee. 2013. “Genetic profiling to predict recurrence of early cervical cancer.” Gynecologic oncology 131 (3):650-654.
Liu, Shu-Chen, Ngan-Ming Tsang, Wen-Che Chiang, Kai-Ping Chang, Chuen Hsueh, Ying Liang, Jyh-Lyh Juang, Kai-Ping N Chow, and Yu-Sun Chang. 2013. “Leukemia inhibitory factor promotes nasopharyngeal carcinoma progression and radioresistance.” The Journal of clinical investigation 123 (12):5269-5283.
Metcalfe, S M. 2011. “LIF in the regulation of T-cell fate and as a potential therapeutic.” Genes & Immunity 12 (3):157-168.
Peng, Meixi, Dan Yang, Yixuan Hou, Shuiqing Liu, Maojia Zhao, Yilu Qin, Rui Chen, Yong Teng, and Manran Liu. 2019. “Intracellular citrate accumulation by oxidized ATM-mediated metabolism reprogramming via PFKP and CS enhances hypoxic breast cancer cell invasion and metastasis.” Cell death & disease 10 (3):1-16.
Peters III, William A, P Y Liu, Rolland J Barrett, Richard J Stock, Bradley J Monk, Jonathan S Berek, Luis Souhami, Perry Grigsby, William Gordon Jr, and David S Alberts. 2000. “Concurrent chemotherapy and pelvic radiation therapy compared with pelvic radiation therapy alone as adjuvant therapy after radical surgery in high-risk early-stage cancer of the cervix.” Obstetrical & Gynecological Survey 55 (8):491-492.
Qi, Yifei, and Ren Xu. 2018. “Roles of PLODs in collagen synthesis and cancer progression.” Frontiers in cell and developmental biology 6:66.
Rose, Peter G, Brian N Bundy, Edwin B Watkins, J Tate Thigpen, Gunther Deppe, Mitchell A Maiman, Daniel L Clarke-Pearson, and Sam Insalaco. 1999. “Concurrent cisplatin-based radiotherapy and chemotherapy for locally advanced cervical cancer.” New England Journal of Medicine 340 (15):1144-1153.
Sedlis, Alexander, Brian N Bundy, Marvin Z Rotman, Samuel S Lentz, Laila I Muderspach, and Richard J Zaino. 1999. “A randomized trial of pelvic radiation therapy versus no further therapy in selected patients with stage IB carcinoma of the cervix after radical hysterectomy and pelvic lymphadenectomy: A Gynecologic Oncology Group Study.” Gynecologic oncology 73 (2):177-183.
Sheta, Razan, Magdalena Bachvarova, Elizabeth Macdonald, Stephane Gobeil, Barbara Vanderhyden, and Dimcho Bachvarov. 2019. “The polypeptide GALNT6 displays redundant functions upon suppression of its closest homolog GALNT3 in mediating aberrant O-glycosylation, associated with ovarian cancer progression.” International journal of molecular sciences 20 (9):2264.
Team, R Core. 2013. “R: A language and environment for statistical computing.”
Vistad, Ingvild, Sophie D Fosså, and Alv A Dahl. 2006. “A critical review of patient-rated quality of life studies of long-term survivors of cervical cancer.” Gynecologic oncology 102 (3):563-572.
Wang, Hua, Shu-Wei Li, Wei Li, and Hong-Bing Cai. 2019. “Elastic Net-Based Identification of a Multigene Combination Predicting the Survival of Patients with Cervical Cancer.” Medical Science Monitor: International Medical Journal of Experimental and Clinical Research 25:10105.
Wong, Yick Fu, Zachariah E Selvanayagam, Nien Wei, Joseph Porter, Ragini Vittal, Rong Hu, Yong Lin, Jason Liao, Joe Weichung Shih, and Tak Hong Cheung. 2003. “Expression genomics of cervical cancer: molecular classification and prediction of radiotherapy response by DNA microarray.” Clinical cancer research 9 (15):5486-5492.

I claim:

1. A method of staging cervical carcinoma in a patient in need thereof, comprising:
determining RNA levels of two or more of the genes of the subject selected from the group consisting of EGLN1, CD46, PLOD1, QSOX1, TM2D1, PEAR1, FKBP9, NRP1, GALNT2, TMED4, KIRREL, LAMC1, SDF4, COPA, FNDC3A, GALNT3, PLK1S1, ANGPTL4, APCDD1L, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145, and subcombinations thereof;
generating machine learning models by using expression data of the two or more genes from randomly selected subsets of patients with cervical cancer;
computing the transcriptomic risk score for each machine learning models and the survival differences between patients with high and low transcriptomic risk score; and
stratifying patients into high, medium or low survivability groups using plurality of voting by the selected models with excellent prediction power.

2. The method of claim 1, wherein the RNA levels are determined using RT-PCT, microarray, RNAseq or any other techniques.

3. The method of claim 1, wherein the machine learning technique is Ridge regression or any other machine learning or artificial intelligence techniques.

4. The method of claim 1, wherein the sample is cervical tissue, tumor tissue, blood, or urine.

5. A method of estimating cervical carcinoma survival time in a patient in need thereof, comprising:
quantifying RNA gene expression in a sample from the patient of the genes selected from the group consisting of EGLN1, CD46, PLOD1, QSOX1, TM2D1.x, PEAR1, FKBP9, NRP1, GALNT2, TMED4, KIRREL, LAMC1, SDF4, COPA, FNDC3A, GALNT3, PLK1S1, ANGPTL4, APCDD1L.x, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145 and subcombinations thereof; and
computing a transcriptomic risk score for the patient using the multigenic models of claim 1, wherein the higher the transcriptomic risk score, the shorter the survival time.

6. A method of determining and monitoring cervical carcinoma treatment response in a subject in need thereof, comprising:
quantifying RNA gene expression in a sample form the subject, wherein the genes include EGLN1, CD46, PLOD1, QSOX1, TM2D1.x, PEAR1, FKBP9, NRP1, GALNT2, TMED4, KIRREL, LAMC1, SDF4, COPA, FNDC3A, GALNT3, PLK1S1, ANGPTL4, APCDD1L.x, ZNF281, MMS19, GPR27, MTDH, LIF, BRSK1, GLG1, KBTBD2, PFKP, CD59, PLAGL1, PRR12, KBTBD6, GRB10, ZC3H12C, FSD1L, AIMP2, ZNF701, RPS6KA2, TMEM167A, RNF145 and subcombinations thereof;
computing a transcriptomic risk scores for the patient using the multigenic models of claim 1 before and after treatment; and
continuing the treatment if the transcriptomic risk score after treatment is the same or less than the score before treatment.

7. The method of claim 6 further comprising the step of altering the treatment if the expression score during treatment is higher than the score before treatment.

8. The method of claim 7, wherein altering the treatment comprises increasing dosing, or adding an additional therapeutic to the treatment.
